SEQUENCE LISTING (38 sequences)

SEQ ID NO: 1 - DNA, 19 bp, synthetic construct
  source 1..19: mol_type = other DNA
gccttgccctctctacggc

SEQ ID NO: 2 - DNA, 21 bp, synthetic construct
  source 1..21: mol_type = other DNA
tgttggctcccaggtcacttc

SEQ ID NO: 3 - DNA, 22 bp, synthetic construct
  source 1..22: mol_type = other DNA
ggaacccaactagatgcagtga

SEQ ID NO: 4 - DNA, 20 bp, synthetic construct
  source 1..20: mol_type = other DNA
cagaggctccgtggaaaaca

SEQ ID NO: 5 - DNA, 20 bp, synthetic construct
  source 1..20: mol_type = other DNA
gccgtccactcgactatcac

SEQ ID NO: 6 - DNA, 21 bp, synthetic construct
  source 1..21: mol_type = other DNA
tgaggaggttgtcaaaggcat

SEQ ID NO: 7 - DNA, 20 bp, synthetic construct
  source 1..20: mol_type = other DNA
tctgagggcttcgacaccta

SEQ ID NO: 8 - DNA, 20 bp, synthetic construct
  source 1..20: mol_type = other DNA
tcattgccggcgcattttag

SEQ ID NO: 9 - DNA, 20 bp, synthetic construct
  source 1..20: mol_type = other DNA
gataacggaggctgggatgc

SEQ ID NO: 10 - DNA, 21 bp, synthetic construct
  source 1..21: mol_type = other DNA
agtcttcagagacagccagga

SEQ ID NO: 11 - DNA, 22 bp, synthetic construct
  source 1..22: mol_type = other DNA
aggcggttgtagaagagtttcg

SEQ ID NO: 12 - DNA, 19 bp, synthetic construct
  source 1..19: mol_type = other DNA
acccaccgaaaaccagagc

SEQ ID NO: 13 - DNA, 20 bp, synthetic construct
  source 1..20: mol_type = other DNA
aagtctagagccaccgtcca

SEQ ID NO: 14 - DNA, 19 bp, synthetic construct
  source 1..19: mol_type = other DNA
cagtctggctgccaatcca

SEQ ID NO: 15 - DNA, 20 bp, synthetic construct
  source 1..20: mol_type = other DNA
gtaacccgttgaaccccatt

SEQ ID NO: 16 - DNA, 20 bp, synthetic construct
  source 1..20: mol_type = other DNA
ccatccaatcggtagtagcg

SEQ ID NO: 17 - DNA, 20 bp, synthetic construct
  source 1..20: mol_type = other DNA
ggacgagctgccctatgatg

SEQ ID NO: 18 - DNA, 20 bp, synthetic construct
  source 1..20: mol_type = other DNA
tttcagcccctttgcactca

SEQ ID NO: 19 - DNA, 21 bp, synthetic construct
  source 1..21: mol_type = other DNA
gtgttgcaagcaggatccaaa

SEQ ID NO: 20 - DNA, 20 bp, synthetic construct
  source 1..20: mol_type = other DNA
ccagtgcttcaacccacaac

SEQ ID NO: 21 - DNA, 20 bp, synthetic construct
  source 1..20: mol_type = other DNA
cgtggagacgggaaagaagt

SEQ ID NO: 22 - DNA, 20 bp, synthetic construct
  source 1..20: mol_type = other DNA
gctgccccagtttttgatcc

SEQ ID NO: 23 - DNA, 20 bp, synthetic construct
  source 1..20: mol_type = other DNA
tgcgggaacacaacagagta

SEQ ID NO: 24 - DNA, 20 bp, synthetic construct
  source 1..20: mol_type = other DNA
aacaactgctcatcacccca

SEQ ID NO: 25 - DNA, 21 bp, synthetic construct
  source 1..21: mol_type = other DNA
agacgtgaaagactgcctcag

SEQ ID NO: 26 - DNA, 19 bp, synthetic construct
  source 1..19: mol_type = other DNA
ttggtcctggttgcccact

SEQ ID NO: 27 - DNA, 21 bp, synthetic construct
  source 1..21: mol_type = other DNA
tcacacctctgacattcacca

SEQ ID NO: 28 - DNA, 20 bp, synthetic construct
  source 1..20: mol_type = other DNA
ttgtcccgatgatctcccct

SEQ ID NO: 29 - DNA, 22 bp, synthetic construct
  source 1..22: mol_type = other DNA
ttggtccgatgtaactcctctg

SEQ ID NO: 30 - DNA, 22 bp, synthetic construct
  source 1..22: mol_type = other DNA
agaagtcgccatgctccttaat

SEQ ID NO: 31 - DNA, 20 bp, synthetic construct
  source 1..20: mol_type = other DNA
gcagccatggcagaagtacc

SEQ ID NO: 32 - DNA, 24 bp, synthetic construct
  source 1..24: mol_type = other DNA
agtcatcctcattgccactgtaat

SEQ ID NO: 33 - DNA, 22 bp, synthetic construct
  source 1..22: mol_type = other DNA
tagcccatgttgtagcaaaccc

SEQ ID NO: 34 - DNA, 21 bp, synthetic construct
  source 1..21: mol_type = other DNA
ttatctctcagctccacgcca

SEQ ID NO: 35 - DNA, 20 bp, synthetic construct
  source 1..20: mol_type = other DNA
acccccaggagaagattcca

SEQ ID NO: 36 - DNA, 20 bp, synthetic construct
  source 1..20: mol_type = other DNA
gatgccgtcgaggatgtacc

SEQ ID NO: 37 - DNA, 20 bp, synthetic construct
  source 1..20: mol_type = other DNA
gtaacccgttgaaccccatt

SEQ ID NO: 38 - DNA, 20 bp, synthetic construct
  source 1..20: mol_type = other DNA
ccatccaatcggtagtagcg

(19) United States
(12) Patent Application Publication
(10) Pub. No.: US 2026/0137631 A1
(43) Pub. Date: May 21, 2026

(54) COMPOSITIONS OF EXTRACELLULAR VESICLES AND METHOD USING THE SAME

(71) Applicant: Ascension Medical Biotechnology Co., Ltd., Taipei City (TW)

(72) Inventors: Ju-Sheng Shieh, Taipei City (TW); Yu-Tang Chin, Taipei City (TW)

(73) Assignee: Ascension Medical Biotechnology Co., Ltd., Taipei City (TW)

(21) Appl. No.: 19/366,535

(22) Filed: Oct. 23, 2025

Related U.S. Application Data
(60) Provisional application No. 63/710,610, filed on Oct. 23, 2024.

(30) Foreign Application Priority Data
Oct. 22, 2025 (TW) 114140898

(51) Int. Cl. A61K 9/51; A61P 17/02; A61P 29/00; A61P 37/06; B82Y 5/00; C12N 5/00; C12N 5/0775; C12N 15/113

(52) U.S. Cl. CPC A61K 9/5176; A61P 17/02; A61P 29/00; A61P 37/06; C12N 5/0018; C12N 5/0662; C12N 15/113; B82Y 5/00; C12N 2310/141; C12N 2500/34; C12N 2500/76; C12N 2501/999

(57) ABSTRACT

The present disclosure provides a composition comprising primed extracellular vesicles and a biologically or pharmaceutically acceptable carrier, wherein the primed extracellular vesicles comprise an upregulated miRNA, and a fold change of the upregulated miRNA of the primed extracellular vesicles compared to miRNA of naturally occurring extracellular vesicles is greater than 1. The present disclosure further provides a method for preventing, treating, or ameliorating a gynecological or a rheumatological disorder comprising administering the composition of the present disclosure to a subject in need thereof. The present disclosure also provides a method for producing the composition. The method comprises cultivating a stem cell in a culture medium containing a Polygonum multiflorum Thunb extract and resveratrol to obtain a cell culture.

### CROSS REFERENCE TO RELATED APPLICATIONS

This application claims the benefit of U.S. Provisional Application No. 63/710,610, filed on Oct. 23, 2024. The content of the application is incorporated herein by reference.

### BACKGROUND OF THE INVENTION

### 1. Field of the Invention

The present disclosure relates to a composition comprising extracellular vesicles, and more particularly to extracellular vesicles produced by the special process and the use of the extracellular vesicles.

### SEQUENCE LISTING

The present disclosure is being filed along with a Sequence Listing in electronic format. The Sequence Listing is provided as a file entitled ASB-P0005-USA-Sequence Listing, created on Oct. 20, 2025, which is 33.7 kb in size. The information in the electronic format of Sequence Listing is incorporated herein by reference in its entirety.

### 2. Description of the Prior Art

Stem cell-based therapy is a treatment approach that uses stem cells per se to repair, regenerate, rejuvenate, or replace damaged or dysfunctional tissues and organs in a subject in need thereof. Stem cell-based therapy holds potential for regenerative medicine and personalized medicine since stems cells are able to self-renew and develop into various cell types. However, stem cell-based therapy still faces several challenges, including transplant rejection, tumor transformation, limited sources, and ethical issues, which limit its clinical application.

Compared to cells per se, extracellular vesicles (EVs) from the cells offer advantages such as low immunogenicity, non-tumorigenicity, high clinical safety, and low ethical risk. The International Society of Extracellular Vesicles (ISEV) defines EVs as “a collective term for particles secreted by cells that are composed of a lipid bilayer and do not possess the ability to replicate independently, meaning they lack a functional nucleus.” Generally, EVs can be classified into three main categories based on their biogenesis mechanisms and particle sizes: apoptotic bodies (approximately 50-2000 nm in diameter), microvesicles (approximately 150-1000 nm in diameter), and exosomes (approximately 30-150 nm in diameter). Due to the size differences among extracellular vesicles, nomenclature often categorizes them as small EVs (sEVs, <200 nm) or medium/large EVs (>200 nm). Since EVs transport signaling molecules such as mRNA, microRNA (miRNA), circRNA, lncRNA, proteins, DNA fragments, and lipids, facilitating signal transmission between cells through internalization, ligand-receptor interactions, or membrane fusion, they play critical roles in physiological and pathological processes.

Compared to the effects of naïve MSC-derived EVs (i.e., naturally occurring extracellular vesicles by the mesenchymal stem cells), which can vary due to the culture conditions of MSCs, numerous studies have shown that EVs from primed or induced MSCs contain different levels of metabolites that may be involved in therapeutic modulation. However, there is still an unmet need in the art to develop an approach to produce primed EVs or guided EVs from MSCs to provide new pathways and solutions for the treatment of various diseases by enhancing the performance of EVs bioactive molecules and improving the therapeutic effect.

### SUMMARY OF THE INVENTION

In one aspect, the present disclosure provides a composition comprising primed extracellular vesicles and a biologically or pharmaceutically acceptable carrier, wherein the primed extracellular vesicles comprise an upregulated miRNA, and a fold change of the upregulated miRNA of the primed extracellular vesicles compared to miRNA of naturally occurring extracellular vesicles is greater than 1.

In some embodiments, the primed extracellular vesicles of the present disclosure are cultivated from mesenchymal stem cells.

In some embodiments, the mesenchymal stem cells of the present disclosure are selected from the group consisting of avian mesenchymal stem cells (hereinafter referred as, AMSCs), human umbilical cord mesenchymal stem cells (hereinafter referred as, hUC-MSCs), human placental mesenchymal stem cells (hereinafter referred as, hPLAMSCs), adipose-derived mesenchymal stem cells, bone-marrow mesenchymal stem cells, and a combination thereof.

In some embodiments, the fold change of the upregulated miRNA of the primed extracellular vesicles of the present disclosure compared to the miRNA of the naturally occurring extracellular vesicles is greater than 1, and the primed extracellular vesicles are cultivated from avian mesenchymal stem cells or from human mesenchymal stem cells. The upregulated miRNA of the primed extracellular vesicles is selected from the group consisting of miR-22-3p, miR-34a-5p, miR-132a-3p, miR-132a-5p, miR-140-3p, miR-140-5p, miR-143-3p, miR-145-5p, miR-146a-5p, miR-146b-5p, miR-146c-5p, miR-181a-5p, miR-181b-5p, miR-199-3p, miR-199-5p, miR-210-3p, miR-210a-5p, miR-212-5p, miR-221-3p, and a combination thereof.

In some embodiments, the fold change of the upregulated miRNA of the primed extracellular vesicles compared to the miRNA of the naturally occurring extracellular vesicles is greater than 1, and the upregulated miRNA of the primed extracellular vesicles is selected from the group consisting of miR-22-3p, miR-143-3p, miR-181a-5p, miR-181b-5p, and a combination thereof.

In some embodiments, the fold change of the upregulated miRNA of the primed extracellular vesicles of the present disclosure compared to the miRNA of the naturally occurring extracellular vesicles is greater than 2, and the upregulated miRNA of the primed extracellular vesicles is selected from the group consisting of miR-143-3p, miR-181a-5p, miR-181b-5p, and a combination thereof.

In some embodiments, the fold change of the upregulated miRNA of the primed extracellular vesicles of the present disclosure compared to the miRNA of the naturally occurring extracellular vesicles is greater than 3, and the upregulated miRNA of the primed extracellular vesicles is selected from the group consisting of miR-181a-5p, miR-181b-5p, and a combination thereof.

In some embodiments, the fold change of a downregulated miRNA of the primed extracellular vesicles compared to miRNA of naturally occurring extracellular vesicles is less than 1.

In some embodiments, the downregulated miRNA of the primed extracellular vesicles is selected from the group consisting of miR-15a-5p, miR-16-5p, miR-16-1-3p, miR-21-5p, miR-26a-5p, miR-29a-3p, miR-29c-3p, miR-34a-5p, miR-126-3p, miR-142-3p, miR-142-5p, miR-155-5p, miR-182-5p, miR-199a-5p, miR-199b-5p, miR-200a-3p, miR-200b-3p, and a combination thereof.

In some embodiments, the extracellular vesicle of the present disclosure is an apoptotic body, a microvesicle, an exosome, or any combination thereof.

In some embodiments, the extracellular vesicles of the present disclosure comprise a polydisperse population of particles with a diameter ranging from about 30 nm to about 200 nm.

In some embodiments, the composition of the present disclosure is formulated into an injection solution, an intra-articular suspension, a vaginal gel, a hydrogel, or a scaffold composition.

In one aspect, the present disclosure further provides a method for preventing, treating, or ameliorating a gynecological or rheumatological disorder comprising administering the composition of the present disclosure to a subject in need thereof.

In some embodiments, the gynecological disorder is selected from the group consisting of genitourinary syndrome of menopause (GSM), premature ovarian insufficiency (POI), ovarian dysfunction, uterine atrophy, and vaginal epithelial thinning.

In some embodiments, the rheumatological disorder is selected from the group consisting of osteoarthritis (OA), rheumatoid arthritis (RA), systemic lupus erythematosus (SLE), psoriatic arthritis, and Sjögren's syndrome.

In one embodiment, the administering of the present disclosure comprises topical administration.

In one embodiment, the administering of the present disclosure comprises local administration.

In some embodiments, the composition of the present disclosure educes chronic inflammation, oxidative stress, immune dysregulation, or progressive tissue degeneration.

In yet another aspect, the present disclosure further provides a method for producing the composition of the present disclosure and the method comprises cultivating a stem cell in a culture medium containing a Polygonum multiflorum Thunb extract and resveratrol to obtain a cell culture; obtaining the extracellular vesicle from the cell culture; and mixing a biologically or pharmaceutically acceptable carrier with the extracellular vesicle.

In some embodiments, the Polygonum multiflorum Thunb extract comprises 2,3,4′,5-tetrahydroxystilbene-2-O-β-D-glucoside (hereinafter referred as THSG).

In some embodiments, the Polygonum multiflorum Thunb extract of the present disclosure has a concentration of about 0.1 μM to about 50 μM.

In some embodiments, the cultivation of the MSCs of the present disclosure is performed for a time period ranging from 24 hours to 96 hours.

The details of one or more embodiments of the present disclosure are set forth in the description below. Other features or advantages of the present disclosure will be apparent from the following drawings and detailed description of several embodiments, and also from the appended claims.

### BRIEF DESCRIPTION OF THE DRAWINGS

The patent or application file contains at least one drawing executed in color. Copies of this patent or patent application publication with color drawings will be provided by the Office upon request and payment of the necessary fee. The following drawings form part of the present specification and are included to further demonstrate certain aspects of the present disclosure. The present disclosure may be better understood by reference to one or more of these drawings in combination with the detailed description of specific embodiments presented herein.

FIG. 1A shows a small-EV particle-size distribution histogram for naïve hUC-MSC EVs (mean 91 nm; min 43 nm; max 657 nm). FIG. 1B shows small-EV particle-size distribution histograms for hUC-MSC TRV-EVs.

FIG. 2A shows small-EV particle-size distribution histograms for naïve AMSC EVs (mean 110 nm; min 81 nm; max 304 nm). FIG. 2B shows small-EV particle-size distribution histograms for AMSC TRV-EVs (mean 73 nm; min 30 nm; max 417 nm).

FIG. 3A, FIG. 3B, FIG. 3C show representative particle-size distribution and particle count comparing naïve AMSC-sEVs with BPEV-TRV under matched measurement settings. Zeta-potential distributions showing a shift toward more negative surface charge for BPEV-TRV relative to source-matched naïve AMSC-sEVs.

FIG. 4 shows human granulosa cell (hGC) proliferation with dose-matched EVs. CCK-8 after 96 h total exposure. Bars: vehicle (0), naïve EVs (1×10⁸/mL), and BPEV-TRV (1×10⁴, 1×10⁶, 1×10⁸/mL). BPEV-TRV increases proliferation dose-dependently and exceeds naïve EVs at the same particle dose; data shown as mean±s.d., significance as indicated in the graph.

FIG. 5 shows CCK-8 viability after 48 h CTX followed by 48 h EV exposure.

FIG. 6A and FIG. 6B show Annexin V/PI flow-cytometry with gated apoptotic populations and particle-dose conditions indicated. CTX 2 μM for 48 h followed by EV treatment 48 h. Flow plots and quantitation: apoptotic fraction is reduced by BPEV-TRV in a dose-dependent manner; statistics annotated in the panel.

FIG. 7A, FIG. 7B, and FIG. 7C show qRT-PCR for AMH, FSHR, LHCGR following 48 h CTX and 48 h EV exposure, respectively.

FIG. 8A, FIG. 8B, FIG. 8C, and FIG. 8D show qRT-PCR for PCNA, BCL-2, TP53, CASP3 following 48 h CTX and 48 h EV exposure, respectively.

FIG. 9 shows Western blots for CASP8, cleaved CASP9, cleaved CASP3, BAX, BCL-2, and PARP (full-length/cleaved) with β-actin control.

FIG. 10A, FIG. 10B, FIG. 10C, FIG. 10D, and FIG. 10E show densitometric quantification normalized to β-actin.

FIG. 11A and FIG. 11B show AMH immunofluorescence with quantification.

FIG. 12A and FIG. 12B show increased estradiol (E2) and progesterone (P4) secretion by ELISA. FIG. 12C shows qRT-PCR demonstrating higher levels of selected miRNAs (miR-21-5p, miR-22-3p, miR-22-5p, miR-199a-5p) in BPEV-TRV versus naïve AMSC-sEVs.

FIG. 13A and FIG. 13B show VK2 vaginal epithelial cell outcomes showing increased proliferation under particle-normalized dosing and rescue of H₂O₂-injured cells, with BPEV-TRV outperforming naïve AMSC-sEVs at matched doses.

FIG. 14 shows human chondrocyte proliferation after 48-h exposure to naïve EVs or BPEV-TRV from AMSC or hUC-MSC (10⁷-10⁹ particles/mL), showing dose-dependent increases with BPEV-TRV exceeding source-matched naïve controls.

FIG. 15 shows human chondrocyte viability under E. coli LPS challenge, depicting (left) LPS dose-response (0.1-10 μg/mL) and (right) rescue at 1 μg/mL by naïve EVs and BPEV-TRV from AMSC or hUC-MSC, with BPEV-TRV providing greater protection than source-matched naïve EVs.

FIG. 16A to FIG. 16E show chondrocyte wound-closure assay (AMSC source) showing baseline gap (“Before treatment”) and 24-h images under control, naïve EVs (1×10⁷ particles/mL), and BPEV-TRV (10⁷ or 10⁹/mL), with BPEV-TRV accelerating closure relative to controls. Wound gap generated with ibidi Culture-Insert 2 Well (500 μm, silicone insert).

FIG. 17A to FIG. 17F show chondrocyte wound-closure assay (hUC-MSC source) showing baseline gap and 24-h images under control, naïve EVs (1×10⁷ particles/mL), and BPEV-TRV (10⁵, 10⁷, or 10⁹/mL), demonstrating dose-responsive enhancement of closure with BPEV-TRV. Wound gap generated with ibidi Culture-Insert 2 Well (500 μm, silicone insert).

FIG. 18A to FIG. 18H show chondrocyte wound-closure under inflammatory challenge (LPS 1 μg/mL, 24 h) comparing control, LPS alone, naïve EVs (1×10⁷ particles/mL)+LPS, and BPEV-TRV (10⁹/mL)+LPS from AMSC and hUC-MSC sources, with BPEV-TRV mitigating LPS-induced migration impairment. Wound gap generated with ibidi Culture-Insert 2 Well (500 μm, silicone insert).

FIG. 19A to FIG. 19C show LPS-stimulated human chondrocytes (24 h), BPEV-TRV reduces pro-inflammatory transcripts TNF-α, IL-1β, and IL-6 relative to LPS and source-matched naïve EVs, with effects shown for AMSC- and hUC-MSC-derived preparations at indicated particle doses.

FIG. 20A and FIG. 20B show BPEV-TRV lowers iNOS and COX-2 mRNA compared with LPS and naïve EVs, for both AMSC and hUC-MSC sources and particle-matched conditions.

FIG. 21A and FIG. 21B show matrix-homeostasis readouts under LPS (24 h) showing COL2A1 up-regulation and MMP-1 down-regulation with BPEV-TRV versus LPS and naïve EVs, for AMSC- and hUC-MSC-derived preparations at particle-matched doses.

### DETAILED DESCRIPTION

Those skilled in the art will readily observe that numerous modifications and alterations of the present disclosure may be made while retaining the teachings of the disclosure described herein. Accordingly, the embodiments described are intended to cover the modifications and alterations within the scope of the present disclosure, rather than to limit the present disclosure. The scope of the claims therefore should be accorded the broadest interpretation so as to encompass all such modifications and alterations.

In this disclosure, all terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein are defined based on the meaning of the terms together with the descriptions throughout the specification.

As used herein, the singular forms “a,” “an,” and “the” are intended to include the plural forms, unless the context clearly indicates otherwise. The terms “includes,” “including,” “comprises,” and “comprising” are used in either the detailed descriptions and/or the claims, and such terms are intended to be inclusive in a manner of not excluding others, such as other components, materials, steps, etc. The terms “sec,” “min,” and “hr” as used herein are abbreviations of “second,” “minute,” and “hour.” The term “or” is used interchangeably with the term “and/or” unless the context clearly indicates otherwise.

As used herein, the term “comprising,” “comprises” “include,” “including,” “have,” “having,” “contain,” “containing,” and any other variations thereof are intended to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitations.

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of this disclosure, unless the context clearly dictates otherwise.

As used herein, the term “about” generally referring to the numerical value meant to encompass variations of ±20%, ±10%, ±5%, ±1%, ±0.5%, or ±0.1% from a given value or range. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations. Alternatively, the term “about” means within an acceptable standard error of the mean when considered by a person having ordinary skill in the art. Unless otherwise expressly specified, all of the numerical ranges, amounts, values and percentages such as those for quantities of materials, durations of time periods, temperatures, operating conditions, ratios of amounts, and the likes disclosed herein should be understood as modified in all instances by the term “about.”

The numeral ranges used herein are inclusive and combinable, any numeral value that falls within the numeral scope herein could be taken as a maximum or minimum value to derive the sub-ranges therefrom. For example, the numeral range “0.1 μM to 50 μM” includes any sub-ranges between the minimum value of 0.1 μM to the maximum value of 50 μM, such as the sub-ranges from 0.1μ M to 40μ, from 10μ M to 50μ, from 1μ M to 20μ M and so on. In addition, a plurality of numeral values used herein can be optionally selected as maximum and minimum values to derive numerical ranges. For instance, the numerical ranges of 0.5 μM to 5 μM, 0.5 μM to 50 μM, and 5 μM to 50 μM can be derived from the numeral values of 0.5 μM, 5 μM, and 50 μM.

As used herein, “cell” refers to the smallest structural unit of living matter capable of functioning autonomously, consisting of one or more nuclei, cytoplasm, and various organelles, all surrounded by a semipermeable membrane. Cells include all somatic cells obtained or derived from a living or deceased animal body at any stage of development as well as germ cells, including sperm and eggs (animal reproductive body consisting of an ovum or embryo together with nutritive and protective envelopes). Included are both general categories of cells: prokaryotes and eukaryotes. The cells contemplated for use in the present disclosure include all types of cells from all organisms in all kingdoms: plans, animals, protists, fungi, archaebacteria and eubacteria. Stem cells are cells capable, by successive divisions, of producing specialized cells on many different levels. For example, hematopoietic stem cells produce both red blood cells and white blood cells. From conception until death, humans contain stem cells, but in adults their power to differentiate is reduced.

As used herein, the term “derived,” when referring to a biological sample, indicates the sample being obtained from the stated source at some point in time. For example, a biological sample derived from an organism can represent a primary biological sample obtained directly from the organism (i.e., unmodified), or can be modified, e.g., by introduction of a recombinant vector, by culturing under particular conditions, or immortalization.

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

As used herein, the term “biologically acceptable carrier” or “pharmaceutically acceptable carrier” refers to a biologically acceptable or pharmaceutically acceptable material, vehicle, or composition, such as a solid or liquid filler, binder, diluent, preservative, biocompatible solvent, disintegrating agent, lubricant, suspending agent, flavoring agent, encapsulating material, thickening agent, acid, surfactant, complexation agent, wetting agent, or any combination thereof. In some embodiments, each component is “pharmaceutically acceptable” in the sense of being compatible with the other ingredients of a pharmaceutical formulation, and suitable for use in contact with the organ or tissue of a subject without excessive toxicity, allergic response, irritation, immunogenicity, or other complications or problems. See, e.g., Remington: The Science and Practice of Pharmacy, 22nd ed.; Allen Ed.: Philadelphia, PA, 2012; Handbook of Pharmaceutical Excipients, 7th ed.; Rowe et al., Eds.; The Pharmaceutical Press and the American Pharmaceutical Association: 2012; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

As used herein, the term “cosmetically acceptable carrier” refers to all carriers and/or excipients and/or diluents conventionally used in topical cosmetic compositions such as in particular in skin care preparations. Common examples include topical vehicles like creams and lotions, natural carrier oils, and advanced systems like liposomes that enhance ingredient penetration and stability. The right carrier is chosen based on the specific product, desired texture, and target skin conditions.

As used herein, the term “effective amount” refers to the amount of an active agent or a pharmaceutical composition that is sufficient to bring about an effect on treating, preventing, or ameliorating a disorder, disease, or condition of a subject in need thereof. The effective amount may vary by a person ordinarily skilled in the art, depending on excipient usage, routes of administration, the possibility of co-usage with other therapeutic treatment, or the condition to be treated, but the present disclosure is not limited thereto.

As used herein, the term “administer,” “administering” or “administration” refer to the placement of an active ingredient into a subject by a method or route which results in at least partial localization of the active ingredient at a desired site to produce the desired effect. For example, the active ingredient of the present disclosure may be administered to a subject by oral administration, injection, subcutaneous administration, intramuscular administration, topical administration, or nasal administration, but the present disclosure is not limited thereto.

As used herein, the term “treat,” “treating,” or “treatment” refers to obtaining a desired pharmacologic and/or physiologic effect. The effect may be prophylactic in terms of completely or partially preventing a disease or symptoms or conditions thereof or may be therapeutic in terms of completely or partially curing, alleviating, relieving, remedying, or ameliorating a disease or an adverse effect attributable to the disease or symptoms or conditions thereof.

As used herein, the term “prevent,” “preventing,” or “prevention” does not require that the disease state be completely thwarted. Rather, as used herein, the term “prevent,” “preventing,” or “prevention” refers to the ability of the skilled artisan to identify a population that is susceptible to disorders, such that administration of the extracellular vesicles of the present disclosure may occur prior to onset of a disease. The term does not imply that the disease state is completely avoided.

As used herein, the terms “patient” and “subject” are used interchangeably. The term “subject” refers to a mammal. The mammal includes, but not limited to, humans, non-human primates, canines, felines, murines, bovines, equines, porcines, sheeps, deers, wolfs, foxes, and rabbits.

As used herein, the term “primed extracellular vesicles” or “bio-pulsed extracellular vesicles” may be used interchangeably to refer to extracellular vesicles derived from cells cultured under a defined stimulation process. The cells may include stem cells such as mesenchymal stem cells (MSCs), and the defined stimulation process may include cultivation in a culture medium containing 2,3,5,4′-tetrahydroxystilbene-2-O-β-D-glucoside (THSG) and resveratrol (hereinafter referred to as TRV bio-pulsing), but the present disclosure is not limited thereto.

As used herein, the term “fold change” is defined as primed extracellular vesicles divided by naïve extracellular vesicles on normalized counts.

In one aspect, the present disclosure provides a composition comprising primed extracellular vesicles and a biologically or pharmaceutically acceptable carrier, wherein the primed extracellular vesicles comprise an upregulated miRNA, and a fold change of the upregulated miRNA of the primed extracellular vesicles compared to miRNA of naturally occurring extracellular vesicles is greater than 1.

In some embodiments, the primed extracellular vesicles of the present disclosure are cultivated from mesenchymal stem cells.

In some embodiments, the mesenchymal stem cells of the present disclosure are selected from the group consisting of avian mesenchymal stem cells (hereinafter referred as, AMSCs), human umbilical cord mesenchymal stem cells (hereinafter referred as, hUC-MSCs), human placental mesenchymal stem cells (hereinafter referred as, hPLAMSCs), adipose-derived mesenchymal stem cells, bone-marrow mesenchymal stem cells, and a combination thereof.

In some embodiments, the fold change of the upregulated miRNA of the primed extracellular vesicles of the present disclosure compared to the miRNA of the naturally occurring extracellular vesicles is greater than 1, and the primed extracellular vesicles are cultivated from avian mesenchymal stem cells or from human mesenchymal stem cells. The upregulated miRNA of the primed extracellular vesicles of the present disclosure is selected from the group consisting of miR-22-3p, miR-34a-5p, miR-132a-3p, miR-132a-5p, miR-140-3p, miR-140-5p, miR-143-3p, miR-145-5p, miR-146a-5p, miR-146b-5p, miR-146c-5p, miR-181a-5p, miR-181b-5p, miR-199-3p, miR-199-5p, miR-210-3p, miR-210a-5p, miR-212-5p, miR-221-3p, and a combination thereof.

In some embodiments, the fold change of the upregulated miRNA of the primed extracellular vesicles of the present disclosure compared to the miRNA of the naturally occurring extracellular vesicles is greater than 2, and the primed extracellular vesicles are cultivated from avian mesenchymal stem cells or from human mesenchymal stem cells. The upregulated miRNA of the primed extracellular vesicles of the present disclosure is selected from the group consisting of miR-132a-3p, miR-132a-5p, miR-143-3p, miR-146a-5p, miR-146b-5p, miR-146c-5p, miR-181a-5p, miR-181b-5p, miR-199-3p, miR-199-5p, miR-210a-5p, miR-212-5p, and a combination thereof.

In some embodiments, the fold change of the upregulated miRNA of the primed extracellular vesicles of the present disclosure compared to the miRNA of the naturally occurring extracellular vesicles is greater than 3, and the primed extracellular vesicles are cultivated from avian mesenchymal stem cells or from human mesenchymal stem cells. The upregulated miRNA of the primed extracellular vesicles of the present disclosure is selected from the group consisting of miR-132a-3p, miR-132a-5p, miR-143-3p, miR-146a-5p, miR-146b-5p, miR-146c-5p, miR-181a-5p, miR-181b-5p, miR-199-3p, miR-199-5p, miR-210a-5p, miR-212-5p, and a combination thereof.

In some embodiments, the fold change of the upregulated miRNA of the primed extracellular vesicles of the present disclosure compared to the miRNA of the naturally occurring extracellular vesicles is greater than 6, and the primed extracellular vesicles are cultivated from avian mesenchymal stem cells or from human mesenchymal stem cells. The upregulated miRNA of the primed extracellular vesicles of the present disclosure is selected from the group consisting of miR-132a-5p, miR-143-3p, miR-146a-5p, miR-146c-5p, miR-181a-5p, miR-181b-5p, miR-199-3p, miR-199-5p, miR-210a-5p, miR-212-5p, and a combination thereof.

In some embodiments, the fold change of the upregulated miRNA of the primed extracellular vesicles of the present disclosure compared to the miRNA of the naturally occurring extracellular vesicles is greater than 7, and the primed extracellular vesicles are cultivated from avian mesenchymal stem cells or from human mesenchymal stem cells. The upregulated miRNA of the primed extracellular vesicles of the present disclosure is selected from the group consisting of miR-143-3p, miR-146a-5p, miR-146c-5p, miR-181b-5p, miR-199-3p, miR-199-5p, miR-210a-5p, miR-212-5p, and a combination thereof.

In some embodiments, the fold change of the upregulated miRNA of the primed extracellular vesicles of the present disclosure compared to the miRNA of the naturally occurring extracellular vesicles is greater than 1, and the primed extracellular vesicles are cultivated from both avian mesenchymal stem cells and human mesenchymal stem cells. The upregulated miRNA of the primed extracellular vesicles is selected from the group consisting of miR-22-3p, miR-143-3p, miR-181a-5p, miR-181b-5p, and a combination thereof.

In some embodiments, the fold change of the upregulated miRNA of the primed extracellular vesicles of the present disclosure compared to the miRNA of the naturally occurring extracellular vesicles is greater than 2, and the primed extracellular vesicles are cultivated from both avian mesenchymal stem cells and human mesenchymal stem cells. The upregulated miRNA of the primed extracellular vesicles is selected from the group consisting of miR-143-3p, miR-181a-5p, miR-181b-5p, and a combination thereof.

In some embodiments, the fold change of the upregulated miRNA of the primed extracellular vesicles of the present disclosure compared to the miRNA of the naturally occurring extracellular vesicles is greater than 3, and the primed extracellular vesicles are cultivated from both avian mesenchymal stem cells and human mesenchymal stem cells. The upregulated miRNA of the primed extracellular vesicles is selected from the group consisting of miR-181a-5p, miR-181b-5p, and a combination thereof.

In some embodiments, the fold change of a downregulated miRNA of the primed extracellular vesicles compared to miRNA of naturally occurring extracellular vesicles is less than 1.

In some embodiments, the downregulated miRNA of the primed extracellular vesicles of the present disclosure is selected from the group consisting of miR-15a-5p, miR-16-5p, miR-16-1-3p, miR-21-5p, miR-26a-5p, miR-29a-3p, miR-29c-3p, miR-34a-5p, miR-126-3p, miR-142-3p, miR-142-5p, miR-155-5p, miR-182-5p, miR-199a-5p, miR-199b-5p, miR-200a-3p, miR-200b-3p, and a combination thereof.

In some embodiments, the extracellular vesicle of the present disclosure is an apoptotic body, a microvesicle, an exosome, or any combination thereof. In some embodiments, the extracellular vesicle of the present disclosure is an exosome and/or a small extracellular vesicle.

In some embodiments, the extracellular vesicles of the present disclosure comprise a polydisperse population of particles with a diameter ranging from about 30 nm to about 200 nm.

In some embodiments, the composition of the present disclosure is formulated into an injection solution, an intra-articular suspension, a vaginal gel, a hydrogel, or a scaffold composition.

In one aspect, the present disclosure further provides a method for preventing, treating, or ameliorating a gynecological or rheumatological disorder comprising administering the composition of the present disclosure to a subject in need thereof.

In some embodiments, the gynecological disorder is selected from the group consisting of genitourinary syndrome of menopause (hereinafter referred as GSM), premature ovarian insufficiency (hereinafter referred as POI), ovarian dysfunction, uterine atrophy, and vaginal epithelial thinning.

For genitourinary syndrome of menopause (GSM), in hypoestrogenic states, vulvovaginal epithelium thins with altered pH, reduced lubrication, dyspareunia, and recurrent infection risk. Conventional measures (lubricants/moisturizers, low-dose vaginal estrogen or DHEA, systemic estrogen, ospemifene) manage symptoms for many but are limited by contraindications and incomplete restoration of mucosal architecture; major guidelines report limited evidence for routine use of energy-based devices; durability and safety profiles remain under assessment. The present disclosure is provided to facilitate understanding and is not an admission regarding prior art.

For premature ovarian insufficiency (POI), POI is defined by loss of ovarian function before age 40 with menstrual disturbance and biochemical evidence of ovarian insufficiency; downstream sequelae include infertility and bone/cardiometabolic risk. Hormone therapy addresses hypoestrogenic symptoms but does not directly repair granulosa or stromal compartments; the 2024 ESHRE-led, evidence-based guideline underscores the need for mechanism-guided, fertility-preserving, and tissue-supportive options.

In some embodiments, the rheumatological disorder is selected from the group consisting of osteoarthritis (hereinafter referred as OA), rheumatoid arthritis (hereinafter referred as RA), systemic lupus erythematosus (hereinafter referred as SLE), psoriatic arthritis, and Sjögren's syndrome.

For osteoarthritis (OA), OA involves IL-1B/TNF-α-driven synovitis, oxidative stress, chondrocyte dysfunction, and MMP/ADAMTS-mediated ECM catabolismon-surgical standards of care (education, exercise/weight mgmt, NSAIDs, intra-articular injections, viscosupplements) improve pain and function but do not restore cartilage; the OARSI clinical guideline emphasizes individualized symptom relief within these constraints.

In one embodiment, the administering of the present disclosure comprises topical administration.

In one embodiment, the administering of the present disclosure comprises local administration. In one embodiment, the local administration comprises injection.

In some embodiments, the composition of the present disclosure educes chronic inflammation, oxidative stress, immune dysregulation, or progressive tissue degeneration.

In yet another aspect, the present disclosure further provides a method for producing the composition of the present disclosure and the method comprises cultivating a stem cell in a culture medium containing a Polygonum multiflorum Thunb extract and resveratrol to obtain a cell culture; obtaining the extracellular vesicle from the cell culture; and mixing a biologically or pharmaceutically acceptable carrier with the extracellular vesicle.

In some embodiments, the Polygonum multiflorum Thunb extract comprises 2,3,4′,5-tetrahydroxystilbene-2-O-β-D-glucoside (hereinafter referred as THSG).

In some embodiments, the Polygonum multiflorum Thunb extract of the present disclosure has a concentration of about 0.1 μM to about 50 μM.

In some embodiments, the cultivation of the MSCs of the present disclosure is performed for a time period ranging from 24 hours to 96 hours.

In some embodiments, the biologically or pharmaceutically acceptable carrier can be selected from the group consisting of inert diluents, dispersing and/or granulating agents, surface active agents and/or emulsifiers, disintegrating agents, binding agents, preservatives, buffering agents, lubricating agents, and/or oils.

In one aspect, the present disclosure further provides a use of the composition in manufacture of a medicament for preventing, treating, or ameliorating a gynecological or a rheumatological disorder.

In one aspect, the present disclosure further provides the composition for use in preventing, treating, or ameliorating a gynecological or a rheumatological disorder.

In some embodiments, the composition of the present disclosure may be formulated as a pharmaceutical composition including primed extracellular vesicles of the present disclosure and a pharmaceutically acceptable carrier.

In one aspect, the present disclosure further provides a non-therapeutic method for preventing, treating, or ameliorating a gynecological or a rheumatological disorder including administering the composition of the present disclosure to a subject in need thereof.

In some embodiments, the composition of the present disclosure may be formulated as a cosmetic composition or cosmeceutical composition including primed extracellular vesicles of the present disclosure and a cosmetically acceptable carrier.

Although the present disclosure is illustrated by specific embodiments and optional features, it is understood that modifications and variations of the concepts herein disclosed may be resorted to by those skilled in the art, and that such modifications and variations are considered to be within the scope of the present disclosure.

### Example

Exemplary embodiments according to the present disclosure are further described in the following examples, which should not be construed to limit the scope of the present disclosure. The materials and methods used in the following examples are described in detail below. The materials used in the present disclosure but unannotated herein are commercially available.

Example 1 TRV Activation of hUC-MSCs to Produce Extracellular Vesicles and Comprehensive miRNA Profiling

### 1. Purpose and Scope

This example discloses a reproducible method to produce extracellular vesicles (EVs) from human umbilical cord mesenchymal stem cells (hUC-MSCs) following TRV activation with botanical polyphenols.

### 2. Cell Source, Qualification, and Expansion

Human umbilical cord mesenchymal stem cells (hUC-MSCs) were obtained from an accredited biobank under informed donor consent and expanded under standard culture conditions. Cells were expanded in Dulbecco's Modified Eagle Medium (DMEM; Gibco) supplemented with 10% exosome-depleted fetal bovine serum (System Biosciences) and 1% penicillin-streptomycin at 37° C. in a humidified atmosphere containing 5% CO₂; for EV collection, cultures were switched to chemically defined, EV-depleted serum-free medium for 24-72 h under TRV exposure. For all experiments, cells between passage 3 and passage 6 were used. Flow cytometry confirmed that >95% of the cells expressed CD73, CD90, and CD105, while <2% expressed CD34, CD45, and HLA-DR.

### 3. TRV Bio-Pulsing and Naïve Control

When cultures reached approximately 70-80% confluence, the medium was replaced with serum-free DMEM containing TRV bio-pulsing agents. The TRV formulation consisted of botanical polyphenols, specifically 2,3,5,4′-tetrahydroxystilbene-2-O-β-D-glucoside (hereinafter referred as THSG) and resveratrol (hereinafter referred as TRV). The effective concentration range of THSG was 0.1-50 μM, and resveratrol 0.1-50 μM. In one embodiment, THSG (10 μM) and resveratrol (5 μM) were applied. Cell viability following TRV exposure remained above 95% as determined by CCK-8 assay, indicating no cytotoxic effect. Cells were incubated for 24-72 hours to allow secretion of TRV-activated extracellular vesicles (TRV-EVs).

For comparative analysis, naïve hUC-MSC-derived EVs were prepared under identical conditions, except that cells were cultured in serum-free DMEM without TRV stimulation. Conditioned medium was collected after 48 hours and processed in parallel with TRV-EVs using identical purification steps.

### 4. EV Harvest, Purification, and Stabilization

Conditioned media (hereinafter referred as CM) from TRV-treated and naïve cultures were processed identically as follows: clarified by sequential centrifugation at 300×g for 10 min, 2,000 ×g for 20 min, and 10,000 ×g for 30 min at 4° C.; passed through a 0.22 μm PES sterile filter; and concentrated by tangential flow filtration (TFF) using a 100 kDa MWCO Cytiva hollow-fiber module operated at a transmembrane pressure of 0.5-0.8 bar and a cross-flow of 25-40 mL/min, with diafiltration against PBS for 5-8 diavolumes to achieve a 10-15×concentration. The TFF retentate was then polished by size-exclusion chromatography using an IZON qEV70 SEC column pre-equilibrated with PBS, and EV-enriched fractions were pooled for downstream processing. The retentate was dispensed into depyrogenated vials and lyophilized, then stoppered under vacuum and stored at 2-8° C. For use, vials were reconstituted with PBS to the original CM-equivalent volume, yielding particle recovery typically exceeding 85%.

### 5. EV Characterization (Human Source Only)

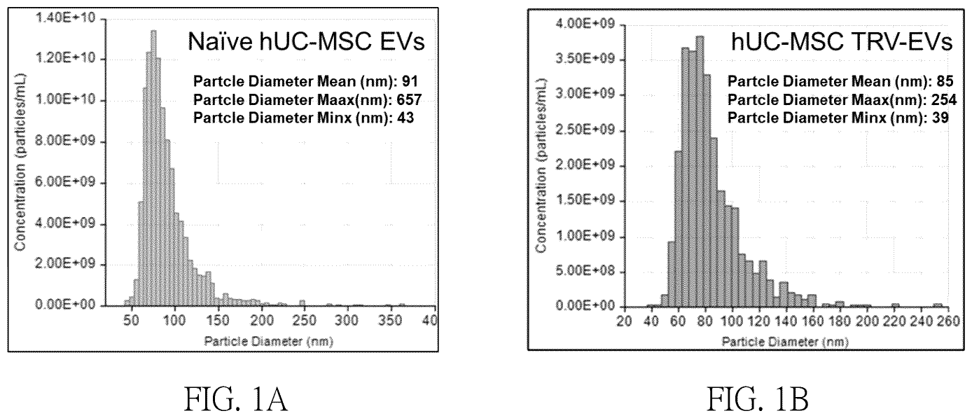

TRPS was performed on an Izon Exoid using CPC-100 calibration beads and an NP150 pore (stretch 47-52), with identical voltage/pressure settings for naïve and TRV-EV runs (FIG. 1A and FIG. 1B). Naïve hUC-MSC EVs showed a nanoscale distribution with a mean diameter of 91 nm (detected min 43 nm, max 657 nm) and a modal peak near ˜90 nm with a sparse high-diameter tail. TRV-activated hUC-MSC EVs remained in the same size class, with a mean of 85 nm (min 39 nm, max 254 nm), a modal region around 70-85 nm, and a modest higher-diameter tail. These measurements indicate that TRV activation preserves the small-EV profile while slightly shifting the central tendency toward smaller diameters under matched instrument conditions. Transmission electron microscopy of carbon-coated grids with uranyl acetate negative staining revealed round, bilayer vesicles in both preparations. Western blots detected CD9, CD63, and CD81, with calnexin undetectable in both TRV-EV and naïve EV samples.

6. miRNA Extraction, Library Preparation, Sequencing, and Statistics

Total RNA from TRV-EVs and naïve EVs (biological n=3 per group) was extracted (exoRNeasy Maxi, Qiagen). RNA quality was checked on Bioanalyzer 2100. Small-RNA libraries were prepared to manufacturer instructions and sequenced on an Illumina platform (paired-end; 8-12 M reads/sample). Adapters were trimmed; reads with Phred <30 were discarded. Mapping to hg38 was performed with standardized parameters; spike-in controls were used to monitor technical variance. Differential expression used DESeq2 with Benjamini-Hochberg correction; adjusted p<0.05 was considered significant. Unless otherwise noted, fold-change (FC) denotes TRV/naïve on normalized counts.

### 7. Differential Hsa-miR Profile

TABLE 1
Differential microRNA profile for hUC-MSC-derived BPEV-TRV versus naïve
EVs, listing read counts, TRV/naïve fold-change, functional significance
related to GSM/POI and steroidogenesis (E2/P4), and mechanism annotations. Table
1 shows an upregulated hsa-miR profile (TRV/Naïve fold-change >1).

| | Naïve | TRV | FC | | |
|---|---|---|---|---|---|
| miRNA | counts | counts | (TRV/Naïve) | Significance | Functional note |
| hsa-miR-181a-5p | 91 | 323 | 3.55 | POI | Follicular development; |
| | | | | | immune microenvironment |
| | | | | | tuning |
| hsa-miR-100-5p | 140 | 745 | 5.32 | POI | mTOR/FGF pathways; follicle |
| | | | | | maturation |
| hsa-miR-181b-5p | 41 | 143 | 3.49 | POI | Follicular development; |
| | | | | | microenvironment tuning |
| hsa-miR-146a-5p | 2 | 81 | 40.50 | GSM | IRAK/TRAF suppression; |
| | | | | (Suppressor-type) | anti-inflammatory |
| hsa-miR-143-3p | 4,977 | 14,413 | 2.90 | POI | Granulosa differentiation; |
| | | | | | follicular dynamics |
| hsa-miR-146b-5p | 3 | 16 | 5.33 | GSM | Anti-inflammatory signaling |
| | | | | (Suppressor-type) | |
| hsa-miR-24-3p | 444 | 573 | 1.29 | GSM, E2/P4 | Pro-survival under |
| | | | | | epithelial/granulosa stress |
| hsa-miR-155-5p | 530 | 609 | 1.15 | POI (Protective | Protective modulation of |
| | | | | downregulation) | ovarian inflammation |
| hsa-miR-22-3p | 304 | 349 | 1.15 | POI, E2/P4 | SIRT1/ER regulation; ovarian |
| | | | | | metabolic homeostasis |
| hsa-miR-199a-3p | 1,207 | 1,522 | 1.26 | GSM, POI | ECM remodeling; |
| | | | | | anti-inflammatory |
| has-miR-199b-3p | 1,207 | 1,522 | 1.26 | GSM, POI | ECM remodeling; |
| | | | | | anti-inflammatory |
| hsa-miR-145-5p | 56 | 92 | 1.64 | POI (Indirect) | Cytoskeletal/contractile |
| | | | | | program (indirect POI support) |
| hsa-miR-27b-3p | 107 | 117 | 1.09 | E2/P4 | Lipid metabolism & |
| | | | | | mitochondrial support; |
| | | | | | promotes steroidogenesis |

TABLE 2
Differential microRNA profile for hUC-MSC-derived BPEV-TRV versus naïve
EVs, listing read counts, TRV/naïve fold-change, functional significance
related to GSM/POI and steroidogenesis (E2/P4), and mechanism annotations. Table
2 shows an downregulated hsa-miR profile (TRV/Naïve fold-change <1).

| | Naïve | TRV | FC | | |
|---|---|---|---|---|---|
| miRNA | counts | counts | (TRV/Naïve) | Significance | Functional note |
| hsa-miR-26a-5p | 3,693 | 3,121 | 0.85 | GSM/POI | Anti-inflammatory; |
| | | | | (Indirect) | pro-differentiation support |
| hsa-miR-142-5p | 203 | 180 | 0.89 | GSM (Protective | Lower immune |
| | | | | downregulation) | infiltration/inflammation |
| hsa-miR-21-5p | 1,608 | 1,245 | 0.77 | GSM, POI, E2/P4 | Anti-apoptotic; PTEN/PDCD4 |
| | | | | | axis; steroidogenesis support |
| hsa-miR-199b-5p | 11 | 8 | 0.73 | GSM, POI | ECM remodeling; |
| | | | | | anti-inflammatory; tissue |
| | | | | | protection |
| hsa-miR-29c-3p | 113 | 93 | 0.82 | GSM (Indirect) | Anti-fibrotic ECM regulation |
| | | | | | (stress reduction) |
| hsa-miR-16-5p | 13,653 | 10,466 | 0.77 | GSM, POI, E2/P4 | Pro-apoptotic via BCL2 |
| | | | | (Protective | repression; reduction supports |
| | | | | downregulation) | survival |
| hsa-miR-199a-5p | 203 | 180 | 0.89 | GSM, POI | ECM remodeling; |
| | | | | | anti-inflammatory; |
| | | | | | anti-inflammatory |
| hsa-miR-29a-3p | 7,544 | 6,313 | 0.84 | GSM (Indirect) | Anti-fibrotic ECM regulation |
| hsa-miR-15a-5p | 77 | 41 | 0.53 | POI, E2/P4 | Relieves BCL2 suppression; |
| | | | | (Protective | protects granulosa cells |
| | | | | downregulation) | |
| hsa-miR-182-5p | 173 | 84 | 0.49 | GSM (Protective | Reduction lowers |
| | | | | downregulation) | stress/oncogenic signaling |
| hsa-miR-142-3p | 39 | 5 | 0.13 | GSM (Protective | Reduced immune |
| | | | | downregulation) | infiltration/inflammation |
| hsa-miR-126-3p | 1,348 | 70 | 0.05 | GSM (Indirect) | Angiogenesis (VEGF axis); |
| | | | | | mucosal perfusion & repair |

FIG. 1A shows small-EV particle-size distribution histograms for naïve hUC-MSC EVs (mean 91 nm; min 43 nm; max 657 nm). FIG. 1B shows small-EV particle-size distribution histograms for hUC-MSC TRV-EVs (mean 85 nm; min 39 nm; max 254 nm). FIG. 1A and FIG. 1B show a small-EV population with a shift toward smaller modal diameters after TRV conditioning.

Example 2 TRV Activation of Avian Mesenchymal Stem Cells (AMSCs) to Produce Extracellular Vesicles and microRNA Profiling

### 1. Purpose and Scope

This example discloses the production of extracellular vesicles (EVs) from avian mesenchymal stem cells (hereinafter referred as AMSCs) following TRV activation with botanical polyphenols, and documents microRNA cargo remodeling by next-generation sequencing (NGS) using gga-miR annotations.

### 2. Materials and Reagents

Basal medium suitable for AMSCs (e.g., high-glucose DMEM or equivalent), exosome-depleted FBS, penicillin-streptomycin, PBS (Mg²⁺/Ca²⁺-free), DMSO (cell-culture grade), TRV polyphenols [representative: 2,3,5,4′-tetrahydroxystilbene-2-O-β-D-glucoside (THSG) and resveratrol], 0.22 μm PES filters, TFF system with 100 kDa MWCO module (Cytiva), Illumina small-RNA library kit and flow cells.

### 3. Cell Source and Qualification

AMSCs were established from fertilized Gallus gallus embryos following institutional guidelines. Master bank P2; working passages P3-P6 were used. Mycoplasma/sterility tests were negative. Flow cytometry confirmed MSC identity (CD73/CD90/CD105 orthologs positive; hematopoietic markers negative), using validated avian-reactive antibodies.

### 4. Culture and TRV Activation

Cells were expanded at 37° C., 5% CO₂ in flasks with basal medium+10% exosome-depleted FBS+1% penicillin-streptomycin. At 70-80% confluence, monolayers were rinsed twice with PBS and switched to serum-free medium containing TRV polyphenols. A representative condition used THSG 10 μM+resveratrol 5 μM (stocks in DMSO; final solvent ≤0.1% v/v) for 24-72 h. Cell viability remained >95% by CCK-8.

### 5. Naïve EV Control

Control EVs were prepared in parallel from matched flasks under identical conditions without TRV. All downstream steps were the same for TRV and naïve preparations.

### 6. EV Harvest and Purification

Conditioned medium (CM) was processed as follows: 300×g 10 min→2,000×g 20 min →10,000×g 30 min (4° C.); 0.22 μm filtration; TFF (100 kDa MWCO) with PBS diafiltration (5-8 diavolumes), transmembrane pressure 0.5-0.8 bar, cross-flow 25-40 mL/min; concentration factor 10-15×. Retentate was collected as EV suspension for analysis or lyophilized as needed.

### A. EV Characterization-TRPS and Zeta Potential

a. TRPS (Izon Exoid, NP150 Pore; Identical Calibration and Instrument Settings for Both Groups)

Naïve AMSC EVs exhibited a predominant nanoscale distribution with a mean particle diameter of 110 nm, with minimum and maximum detected diameters of 81 nm and 304 nm, respectively. The histogram shows a unimodal peak centered near 100-120 nm with a low-abundance long-diameter tail. TRV-activated AMSC EVs (hereinafter referred as AMSC-TRV-EVs) remained within the small-EV size class but displayed a left-shifted central tendency, with a mean particle diameter of 73 nm and minimum and maximum detected diameters of 30 nm and 417 nm, respectively. Under matched settings, the TRV-EV histogram shows a higher peak and broader higher-diameter tail, indicating enhanced vesicle output while preserving the expected nanoscale profile (representative peak concentration on the order of ˜3×10⁹ particles/mL vs. ˜5×10⁸ particles/mL for naïve).

b. Zeta Potential (Exoid Method)

Measured on an Izon Exoid TRPS system under the Zeta potential (Exoid method) conditions described herein. Naïve AMSC EVs showed a distribution concentrated around −20 to −25 mV, with an average near −25 mV. AMSC-TRV-EVs exhibited a more negative distribution, concentrated around −40 to −50 mV, with an average near −45 m V. Overall, TRV activation shifted the zeta potential toward more negative values and yielded a tighter distribution, consistent with increased colloidal stability of the EV preparation.

7. miRNA Extraction, Library Preparation, Sequencing, and Statistics

Total RNA from TRV-EVs and naïve EVs (biological n≥3 per group, where available) was extracted (exoRNeasy or equivalent), assessed on Bioanalyzer 2100, prepared as small-RNA libraries, and sequenced on an Illumina platform (8-12 M reads/sample). Adapters were trimmed; reads with Phred <30 were discarded. Mapping used the Gallus gallus genome with gga-miR annotations. Differential expression employed DESeq2 with Benjamini-Hochberg correction; adjusted p<0.05 was considered significant. Fold-change (FC) is TRV/naïve on normalized counts.

### 8. Differential Gga-miR Profile (Avian Source Only)

TABLE 3
Differential microRNA profile for AMSC-derived BPEV-TRV versus naïve AMSC-sEVs, listing read
counts, TRV/naïve fold-change, functional significance for GSM/POI and E2/P4 support, and mechanism
annotations. Table 3 shows an upregulated gga-miR profile (TRV/Naïve fold-change >1).

| | Naïve | TRV | FC | Significance | Mechanism |
|---|---|---|---|---|---|
| miRNA | counts | counts | (TRV/Naïve) | (context) | (representative) |
| gga-miR-212-5p | 0 | 193 | —* | E2/P4 | CREB-Star axis; classical |
| | | | | | enhancer of steroidogenesis |
| gga-miR-199-3p | 211 | 68,075 | 322.63 | GSM, POI | ECM remodeling; |
| | | | | | anti-inflammatory; tissue |
| | | | | | protection |
| gga-miR-210a-5p | 212 | 28,597 | 134.89 | GSM | Hypoxia-responsive; |
| | | | | | promotes angiogenesis & |
| | | | | | repair |
| gga-miR-181b-5p | 513 | 43,511 | 84.82 | POI | Follicular development; |
| | | | | | immune microenvironment |
| | | | | | tuning |
| gga-miR-146c-5p | 4,906 | 352,859 | 71.92 | GSM | Inhibits |
| | | | | (suppressor-type) | IRAK/TRAF-mediated |
| | | | | | inflammation |
| gga-miR-199-5p | 925 | 59,526 | 64.35 | GSM, POI | ECM remodeling; |
| | | | | | anti-inflammatory |
| gga-miR-100-5p | 1,580 | 38,298 | 24.24 | POI | Follicle maturation via |
| | | | | | mTOR/FGF pathways |
| gga-miR-24-3p | 3,700 | 71,189 | 19.24 | GSM, E2/P4 | Cell survival; protects |
| | | | | | epithelial/granulosa cells |
| gga-miR-143-3p | 9,837 | 183,635 | 18.67 | POI | Granulosa-cell |
| | | | | | differentiation and follicular |
| | | | | | dynamics |
| gga-miR-23b-3p | 7,000 | 93,676 | 13.38 | GSM, E2/P4 | Anti-inflammatory; |
| | | | | | epithelial homeostasis |
| gga-miR-27b-3p | 7,915 | 68,897 | 8.70 | E2/P4 | Lipid & mitochondrial |
| | | | | | support; steroidogenesis |
| gga-miR-21-5p | 210,645 | 1,423,493 | 6.76 | GSM, POI, | Anti-apoptotic; |
| | | | | E2/P4 | PTEN/PDCD4 axis; |
| | | | | | steroidogenesis |
| gga-miR-181a-5p | 15,304 | 98,713 | 6.45 | POI | Follicular development; |
| | | | | | immune microenvironment |
| gga-miR-132a-5p | 34 | 208 | 6.12 | E2/P4 | CREB-Star axis; |
| | | | | | steroidogenesis |
| gga-miR-132a-3p | 30 | 168 | 5.60 | E2/P4 | CREB-Star axis; |
| | | | | | steroidogenesis |
| gga-miR-22-3p | 35,839 | 66,549 | 1.86 | POI, E2/P4 | SIRT1/ER signaling; |
| | | | | | metabolic support |

*undefined due to zero denominator

TABLE 4
Differential microRNA profile for AMSC-derived BPEV-TRV versus naïve AMSC-sEVs, listing read
counts, TRV/naïve fold-change, functional significance for GSM/POI and E2/P4 support, and mechanism
annotations. Table 4 shows a downregulated gga-miR profile (TRV/Naïve fold-change <1).

| | Naïve | TRV | FC | Protective | |
|---|---|---|---|---|---|
| miRNA | counts | counts | (TRV/Naïve) | context | Mechanism (representative) |
| gga-miR-200a-3p | 1,906 | 330 | 0.17 | E2/P4 | Down-regulation stabilizes |
| | | | | | endocrine function; reduces |
| | | | | | EMT/stress signaling |
| gga-miR-200b-3p | 1,052 | 150 | 0.14 | E2/P4 | Reduces EMT/stress; |
| | | | | | endocrine stabilization |
| gga-miR-142-3p | 102,462 | 13,036 | 0.13 | GSM | Lowers immune infiltration |
| | | | | | and inflammation |
| gga-miR-16-5p | 13,600,855 | 1,521,372 | 0.11 | GSM, POI, | Normally pro-apoptotic via |
| | | | | E2/P4 | BCL2 repression; reduction |
| | | | | | supports survival |
| gga-miR-182-5p | 58,482 | 6,673 | 0.11 | GSM | Pro-inflammatory/oncogenic; |
| | | | | | reduction lowers stress |
| gga-miR-142-5p | 22,279 | 2,503 | 0.11 | GSM | Reduced immune |
| | | | | | infiltration/inflammation |
| gga-miR-16-1-3p | 1,751 | 135 | 0.08 | POI, E2/P4 | Relieves BCL2 suppression; |
| | | | | | supports granulosa-cell |
| | | | | | survival |

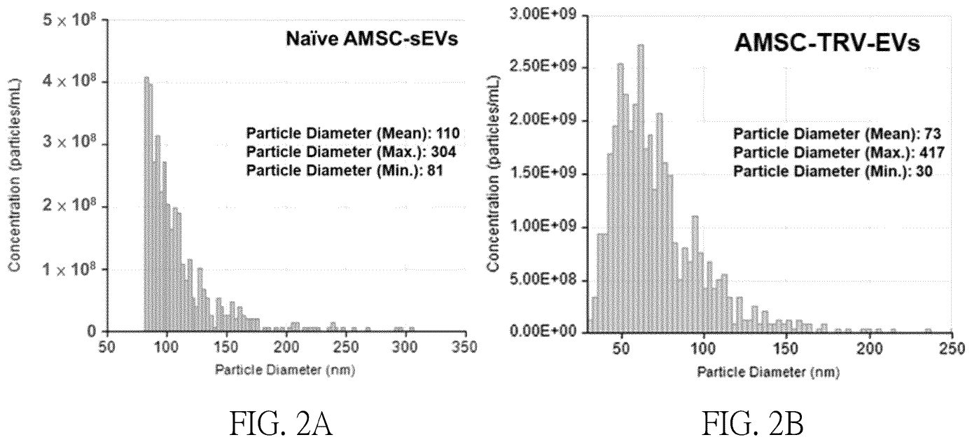

FIG. 2A shows a small-EV particle-size distribution histograms for naïve AMSC EVs (mean 110 nm; min 81 nm; max 304 nm). FIG. 2B shows a small-EV particle-size distribution histograms for AMSC TRV-EVs (mean 73 nm; min 30 nm; max 417 nm). FIG. 2A and FIG. 2B show a small-EV population with a shift toward smaller modal diameters after TRV conditioning.

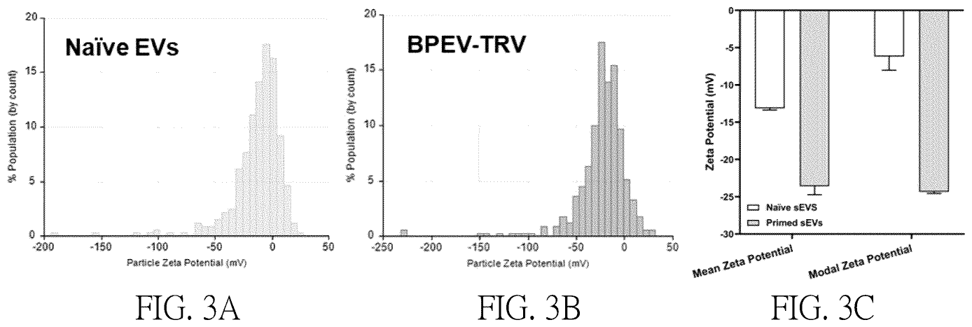

FIG. 3A shows a Zeta potential distribution of Naïve EVs-population centered at approximately −20 to −25 mV. FIG. 3B shows a Zeta potential distribution of BPEV-TRV (AMSC-TRV-EVs)-population centered at approximately −40 to −50 mV. FIG. 3C shows a Bar summary of mean and modal zeta potentials for the two groups, demonstrating that TRV-EVs exhibit a more negative surface charge, consistent with improved dispersion stability.

Example 3 BPEV-TRV (Primed AMSC-sEVs) Restore Granulosa-Cell Function in a Cyclophosphamide POI Model

### 1. Purpose and Scope

This example evaluates BPEV-TRV, i.e., extracellular vesicles (EVs) produced from avian mesenchymal stem cells (AMSCs) under TRV bio-pulsing (priming), in an in vitro POI-like model using human granulosa cells (hGCs). Endpoints cover proliferation, apoptosis, ovarian function-associated gene expression, steroidogenic output, and pathway markers. Naïve AMSC-EVs and vehicle serve as comparators. All EV doses are dose-matched by particle number.

### 2. Materials

hGCs (HGL5 Line) Cultured Per Supplier Guidance.

Cyclophosphamide (CTX) stock in DMSO.

BPEV-TRV and naïve AMSC-EVs prepared as shown in Example 2; lyophilized and reconstituted in PBS.

Assay kits: CCK-8 for viability; Annexin V/PI apoptosis; ELISAs for estradiol (E2) and progesterone (P4).

Reagents for qRT-PCR (targets: AMH, FSHR, LHCGR, PCNA, BCL-2, TP53, CASP3; reference 18S) and immunoblotting (cleaved CASP3, CASP9, PARP, BAX, BCL-2, β-actin).

TABLE 5
Primer sequences for Homo sapiens

| Gene | | | |
|---|---|---|---|
| name | Forward | Backward | Accession No. |
| AMH | GCCTTGCCCTCTCTACGGC | TGTTGGCTCCCAGGTCACTTC | NM_000479.5 |
| FSHR | GGAACCCAACTAGATGCAGTGA | CAGAGGCTCCGTGGAAAACA | M65085.1 |
| LHCGR | GCCGTCCACTCGACTATCAC | TGAGGAGGTTGTCAAAGGCAT | M73746.1 |
| PCNA | TCTGAGGGCTTCGACACCTA | TCATTGCCGGCGCATTTTAG | BC062439.1 |
| BCL-2 | GATAACGGAGGCTGGGATGC | AGTCTTCAGAGACAGCCAGGA | NM_000633.3 |
| CASP3 | AGGCGGTTGTAGAAGAGTTTCG | ACCCACCGAAAACCAGAGC | NM_004346.4 |
| TP53 | AAGTCTAGAGCCACCGTCCA | CAGTCTGGCTGCCAATCCA | NM_000546.5 |
| 18S | GTAACCCGTTGAACCCCATT | CCATCCAATCGGTAGTAGCG | NR_003286 |

Abbreviation:
AMH: anti-Mullerian hormone; FSHR: follicle stimulating hormone receptor; LHCGR: lutropin/choriogonadotropin receptor; PCNA: proliferating cell nuclear antigen; BCL-2: B cell lymphoma/leukemia type 2; CASP3: cysteine-aspartic acid protease (caspase)-3; TP53: tumor protein p53 (p53); 18S: 18S ribosomal RNA.

### Antibodies for AMH Immunofluorescence

### 3. Methods

### 3.1 CTX Injury (POI-Like) Model

hGCs are seeded, serum-restricted, then exposed to CTX 2 μM for 48 h to induce injury.

### 3.2 EV Treatment

After CTX, medium is replaced and cells receive: vehicle; naïve EVs 1×10⁸ particles/mL; or BPEV-TRV at 1×10⁴, 1×10⁶, 1×10⁸ particles/mL for another 48 h. Parallel “no-CTX” cohorts receive the same EV dose set.

### 3.3 Assays.

Proliferation/viability: CCK-8 at 96 h total (or 48 h post-EV).

Apoptosis: Annexin V/PI by flow cytometry and analysis used FSC/SSC singlet gating; apoptotic fraction defined as Annexin V⁺ (±PI). AnnexinV⁺/PI±quadrants with identical voltages across groups.

qRT-PCR: AMH, FSHR, LHCGR; PCNA; BCL-2; TP53; CASP3; ΔΔCt vs. 18S.

Western blot: cleaved CASP3, CASP9, PARP; BAX; BCL-2; normalized to β-actin.

Immunofluorescence (IF): AMH; identical imaging parameters across groups; ImageJ quantitation.

Steroidogenesis: E2 (pg/mL) and P4 (pg/mL) in supernatants by ELISA.

3.4 Statistics. N>6 Wells (qRT-PCR/ELISA/CCK-8), n>3 Blots (WB), Two-Sided ANOVA with Pre-Specified Multiple-Comparison Tests; p<0.05.

### 4. Results (Dose-Matched by Particle Count)

### 4.1 Proliferation

As shown in FIG. 4, relative to vehicle, naïve EVs (1×10⁸/mL) produce a modest but significant increase in hGC proliferation (˜1.2×). BPEV-TRV shows dose-dependent enhancement to roughly ˜1.25×(10⁴), ˜1.4×(10⁶), and ˜1.6×(10⁸), and outperforms naïve EVs at equal particle dose.

### 4.2 Cytoprotection Under CTX

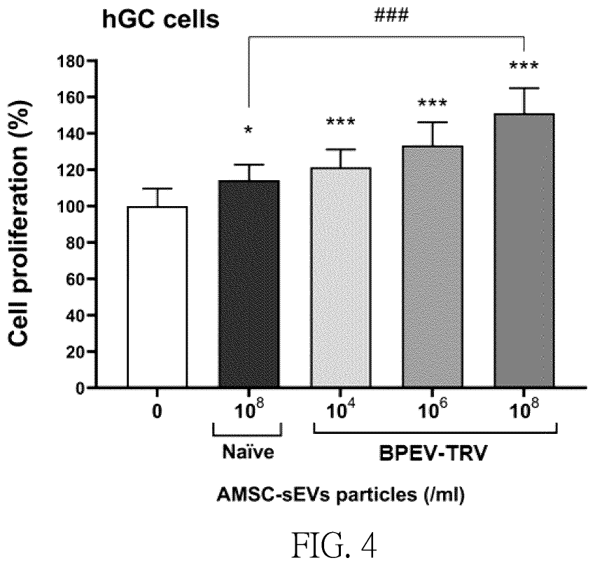

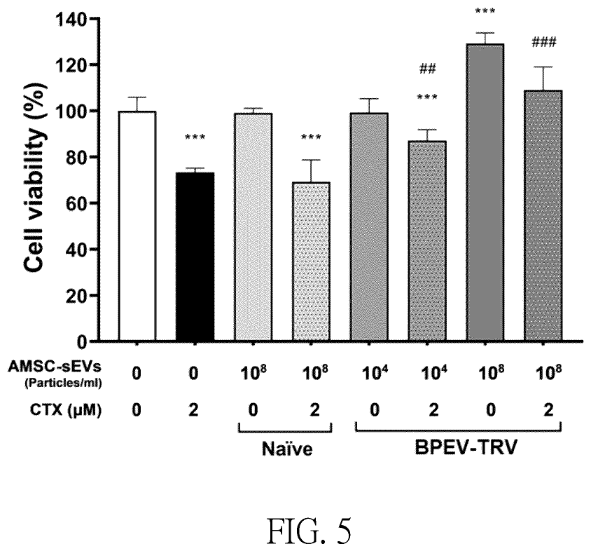

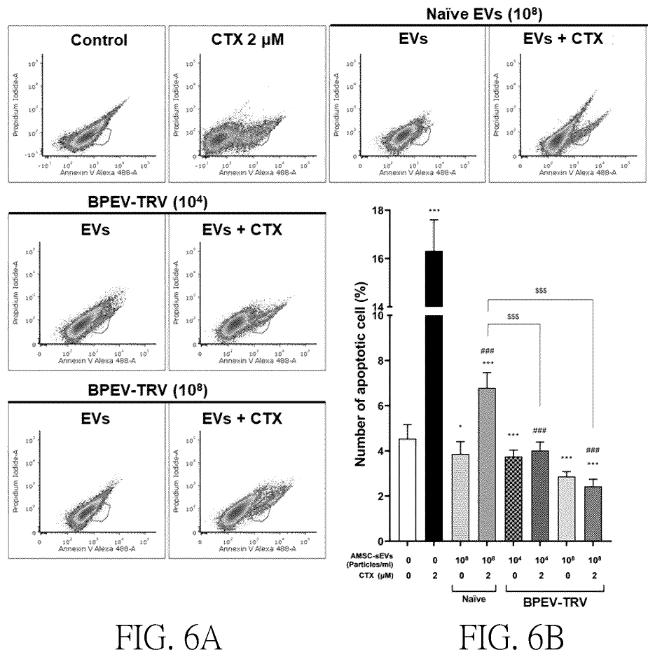

As shown in FIG. 5, FIG. 6A and FIG. 6B, CTX (2 μM) depresses viability and elevates apoptosis. Post-injury treatment with BPEV-TRV restores viability above baseline controls and reduces Annexin V/PI-positive cells substantially (representative drop from the high-teens % with CTX alone to low single-digits at 1×10⁸/mL), outperforming the naïve EV condition at the same dose.

### 4.3 Ovarian Function-Associated Genes

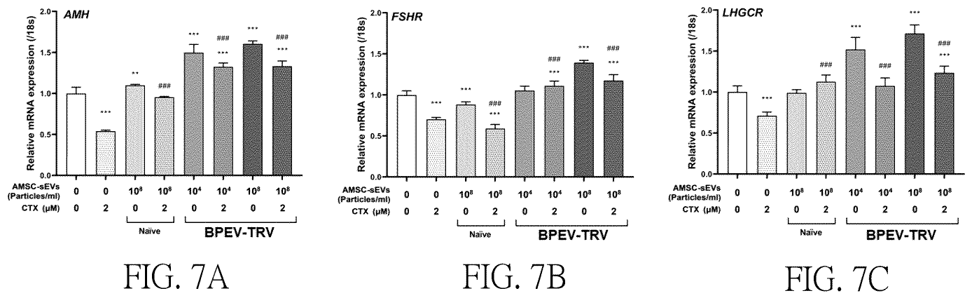

As shown in FIG. 7A, FIG. 7B and FIG. 7C, in the CTX-injured hGC model, BPEV-TRV AMSC-SEVs restore ovarian-function transcripts and rebalance proliferation/apoptosis markers relative to vehicle and naïve AMSC-sEVs. CTX suppresses AMH, FSHR, and LHCGR. BPEV-TRV reverses this suppression in a dose-dependent manner and raises expression toward or above uninjured levels. Naïve EVs restore AMH but are less effective for FSHR/LHCGR.

### 4.4 Proliferative and Apoptosis-Program Genes

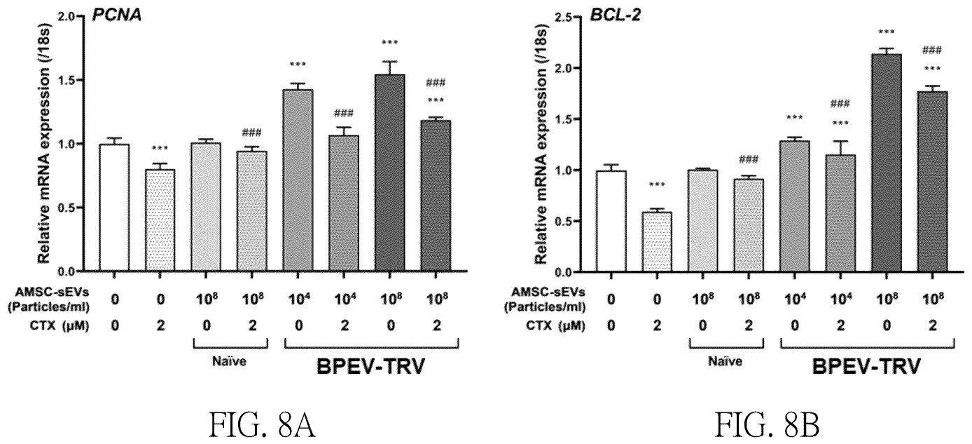

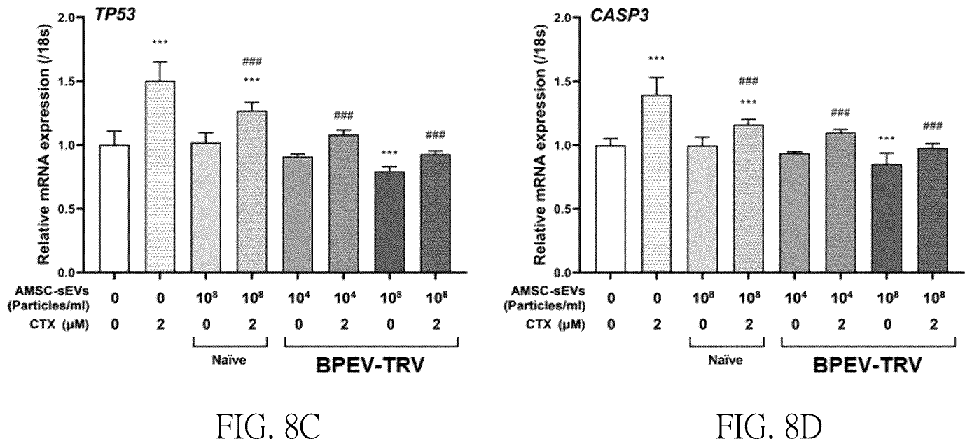

As shown in FIG. 8A, FIG. 8B, FIG. 8C and FIG. 8D, in the CTX-injured hGC model, BPEV-TRV AMSC-SEVs restore ovarian-function transcripts and rebalance proliferation/apoptosis markers relative to vehicle and naïve AMSC-sEVs. BPEV-TRV increases PCNA and BCL-2, while TP53 and CASP3 are reduced versus CTX-only and versus naïve EVs at the same dose.

### 4.5 Intrinsic Apoptosis Pathway Proteins

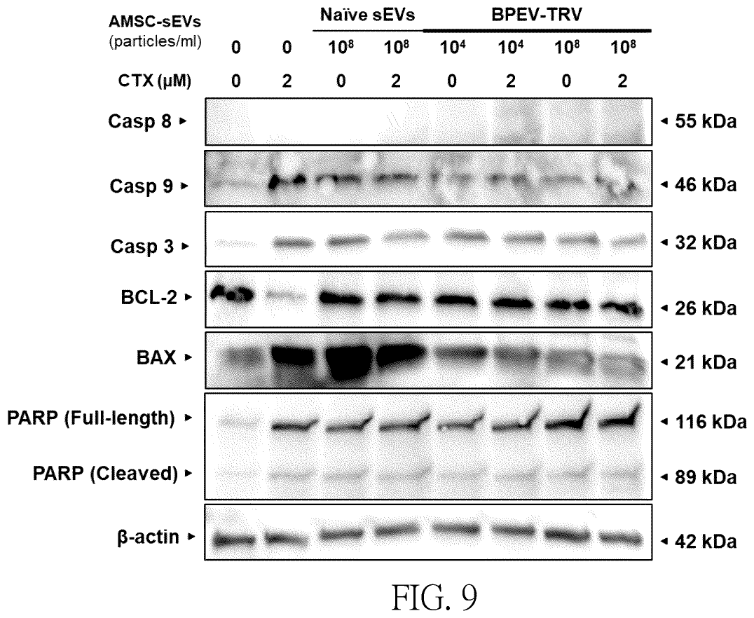

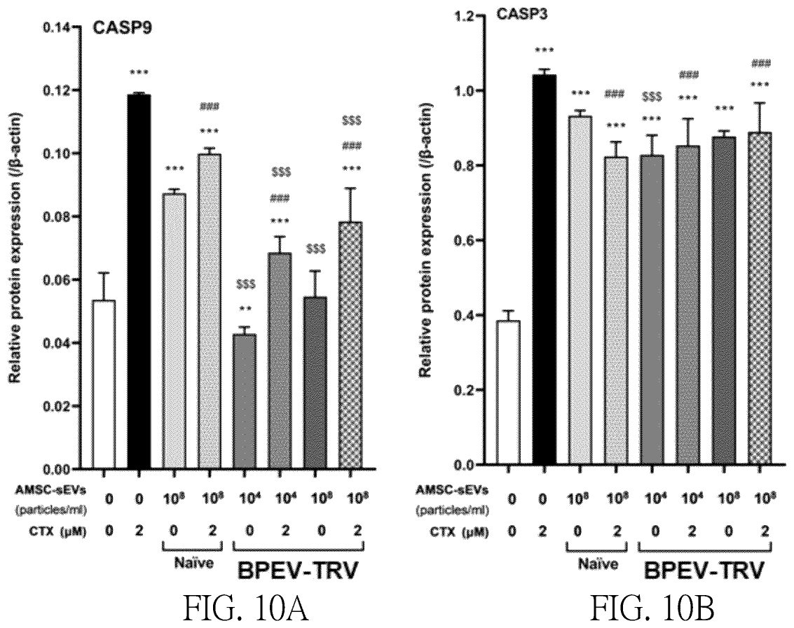

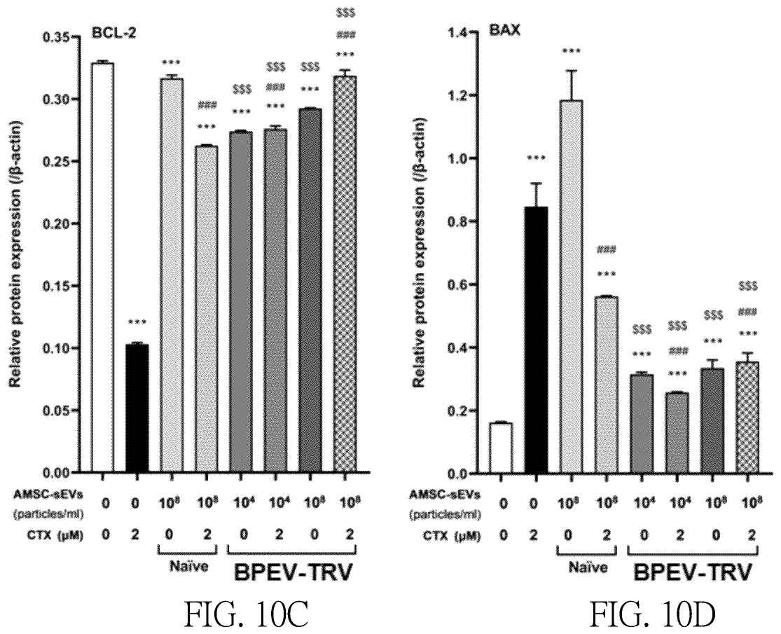

As shown in FIG. 9, FIG. 10A, FIG. 10B, FIG. 10C, FIG. 10D, and FIG. 10E, in CTX-treated hGCs, BPEV-TRV AMSC-sEVs attenuate apoptosis relative to naïve AMSC-SEVs. apoptosis pathway proteins by Western blot. Representative blots and densitometry for cleaved CASP3, cleaved CASP9, cleaved PARP, BAX, BCL-2, and CASP8. BPEV-TRV reduces intrinsic apoptosis markers and elevates BCL-2 versus CTX-only and naïve EVsCTX increases cleaved CASP3, cleaved CASP9, BAX, and cleaved PARP. BPEV-TRV reduces cleaved CASP3/9 and BAX, preserves full-length PARP, and elevates BCL-2; CASP8 remains largely unchanged—consistent with predominant mitochondrial (intrinsic) pathway modulation.

### 4.6 AMH Protein and Steroid Hormones

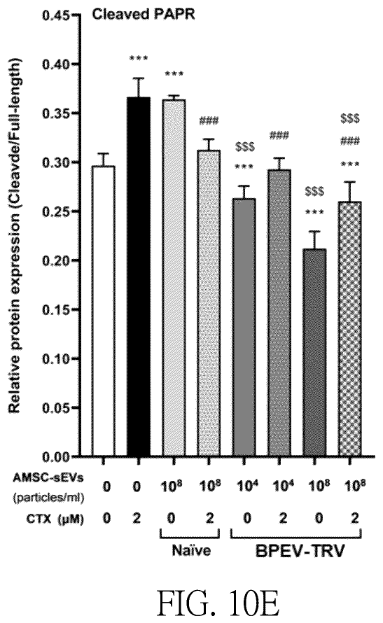

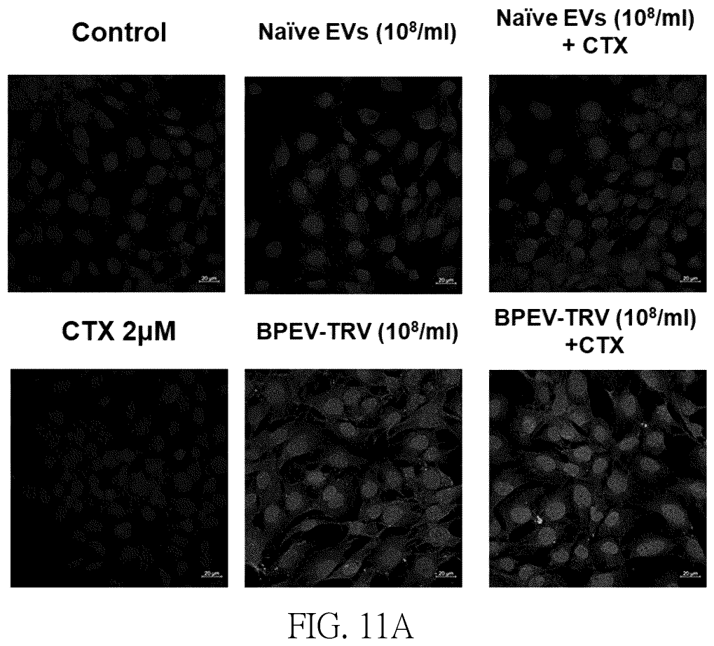

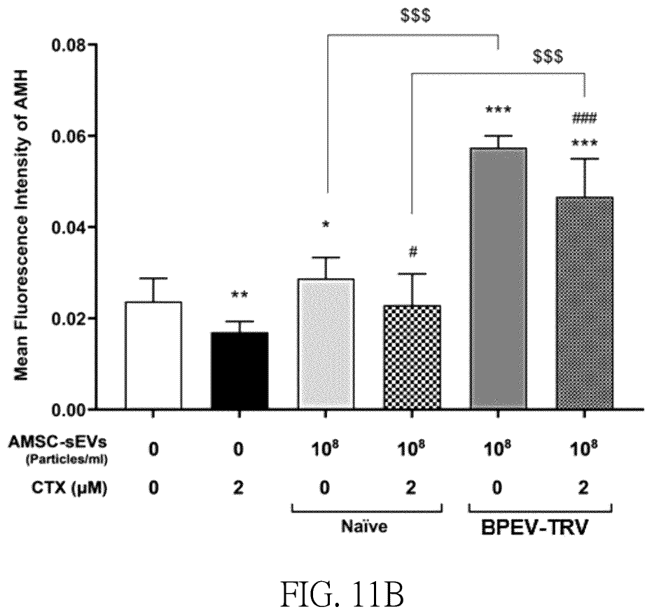

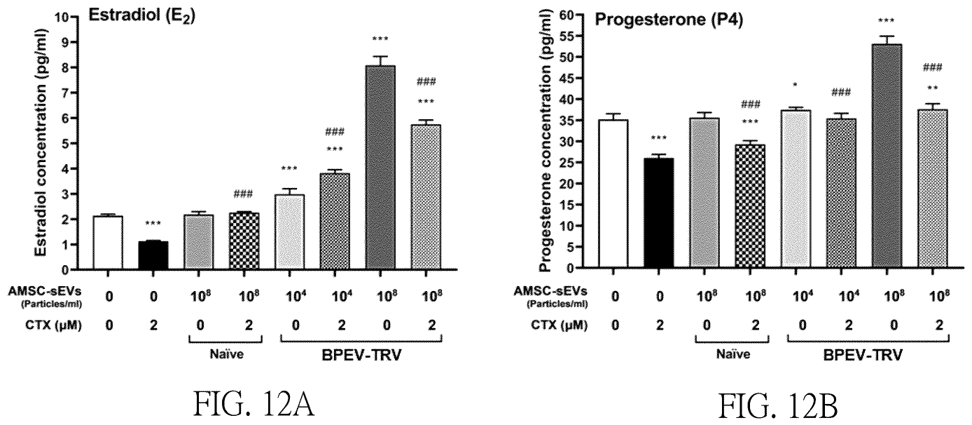

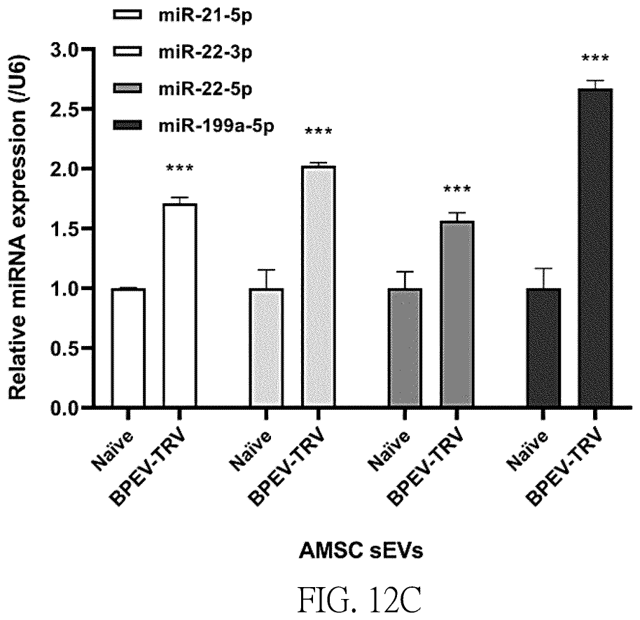

As shown in FIG. 11A and FIG. 11B, in CTX-injured hGCs, BPEV-TRV AMSC-SEVs restore steroidogenic function and correlate with a primed miRNA cargo. BPEV-TRV increases AMH immunofluorescence intensity versus CTX and naïve EVs. As shown in FIG. 12A and FIG. 12B, secreted E2 and P4 rise dose-dependently with BPEV-TRV and are restored from CTX-suppressed levels; naïve EVs show partial restoration.

4.7 EV miRNA Markers

As shown in FIG. 12C, in CTX-injured hGCs, BPEV-TRV AMSC-sEVs restore steroidogenic function and correlate with a primed miRNA cargo. qRT-PCR of BPEV-TRV vs. naïve AMSC-EVs shows higher levels of miR-21-5p, miR-22-3p, miR-22-5p, miR-199a-5p, aligning with the NGS trends disclosed in Example 2 and consistent with anti-apoptotic and steroidogenic support.

### 5. Interpretation

Under particle-matched dosing, BPEV-TRV exceeds naïve AMSC-EVs across proliferation, cytoprotection, restoration of AMH/FSHR/LHCGR, normalization of E2/P4, and repression of intrinsic apoptosis markers. The data support a potency contribution from remodeled miRNA cargo, including the miR-21/22/199 axis, together with enhanced EV physicochemical properties disclosed in Example 2 (smaller mean size and more negative zeta potential). Collectively, BPEV-TRV provides a granulosa-cell protective and steroidogenic effect profile relevant to POI and GSM indications.

Example 4 GSM Application: BPEV-TRV (Primed AMSC-sEVs) Enhance Proliferation and Rescue Oxidative Injury in Human Vaginal Epithelial Cells (VK2/E6E7)

### 1. Purpose and Scope

This example evaluates the efficacy of BPEV-TRV—extracellular vesicles derived from avian mesenchymal stem cells (AMSCs) under TRV bio-pulsing (priming)—in a genitourinary syndrome of menopause (GSM)-relevant in-vitro model using the VK2/E6E7 vaginal epithelial cell line. Outcomes include basal proliferation and rescue of H₂O₂-induced oxidative injury, with naïve AMSC-EVs and vehicle as comparators. EV dosing is normalized by particle number.

### 2. Materials

Cells: VK2/E6E7 (ATCC CRL-2616), authenticated and mycoplasma-free.

Culture medium: Keratinocyte-SFM (serum-free) supplemented per supplier instructions (bovine pituitary extract, EGF, calcium; 1% penicillin-streptomycin).

EVs: BPEV-TRV and naïve AMSC-EVs prepared as shown in Example 2 (AMSC source), purified by TFF (100 kDa MWCO), stored lyophilized and reconstituted in PBS.

Reagents: H₂O₂ (freshly prepared), CCK-8 kit, Annexin V/PI kit (optional), PBS, DMSO (≤0.1% v/v in all wells).

### 3. Methods

### 3.1 Cell Seeding and Culture.

VK2 cells were seeded in 96-well plates (1.0-1.5×10⁴ cells/well) and cultured to 70-80% confluence at 37° C., 5% CO₂.

### 3.2 EV Treatment—Basal Proliferation.

Medium was replaced with fresh supplemented Keratinocyte-SFM containing EVs at the indicated particle concentrations for 48 h:
Vehicle control (PBS);
Naïve AMSC-EVs at 1×10⁸ particles/mL;
BPEV-TRV at 1×10⁴, 1×10⁶, or 1×10⁸ particles/mL.

### 3.3 Oxidative-Injury Rescue.

To model GSM-relevant epithelial stress, cells were pre-injured with H₂O₂ 100 μM for 30 min in serum-free medium, rinsed once with PBS, then treated 12 h with vehicle, naïve EVs (1×10⁸/mL), or BPEV-TRV (1×10⁶ or 1×10⁸/mL). Optional: a co-treatment arm (H₂O₂ present during EV exposure) may be included; results were concordant in pilot runs.

### 3.4 Readouts and Statistics.

CCK-8 absorbance was recorded and reported as % of control.

Each condition used n>6 wells; at least three independent experiments were performed. Data were analyzed by one-way ANOVA with multiple-comparison corrections; p<0.001 thresholds are indicated in the figures.

### 4. Results

### 4.1 Basal Proliferation

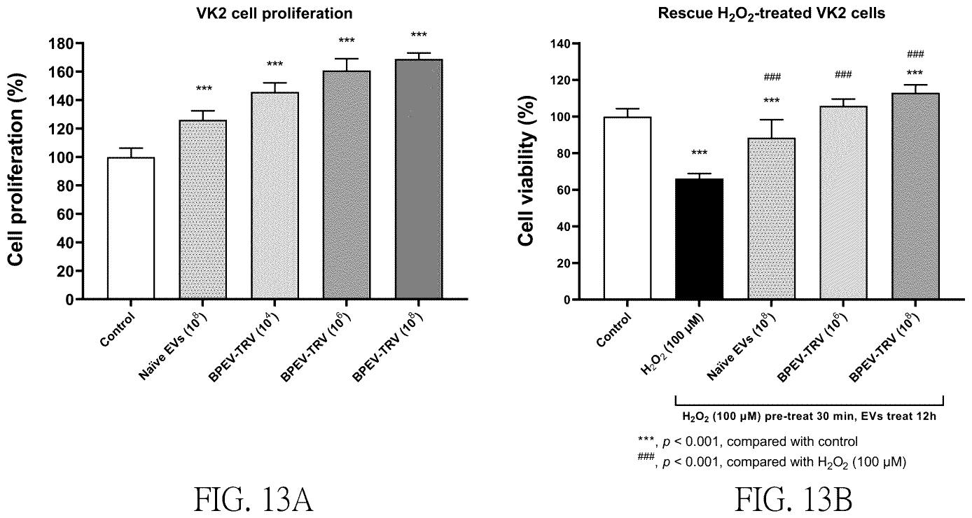

As shown in FIG. 13A, cell proliferation (% of control) after 48 h with vehicle, naïve AMSC-EVs (1×10⁸/mL), or BPEV-TRV (1×10⁴, 1×10⁶, 1×10⁸/mL). BPEV-TRV shows dose-dependent increases and exceeds naïve EVs at matched or lower particle numbers (*** p<0.001 vs. control). Naïve AMSC-EVs (1×10⁸/mL) increased VK2 proliferation above vehicle. BPEV-TRV produced dose-dependent gains; the 1×10⁶ and 1×10⁸/mL groups exceeded the naïve EV condition at equal or lower particle numbers, with significance denoted (*** vs. control).

4.2 H₂O₂ rescue

As shown in FIG. 13B, cell viability (% of control) in H₂O₂ (100 μM)-injured VK2 cells. After 30-min H₂O₂ exposure, EVs were applied for 12 h. Naïve EVs partially restore viability (*** vs. control; ###vs. H₂O₂), whereas BPEV-TRV (1×10⁶, 1×10⁸/mL) confers greater rescue (*** vs. control; ###vs. H₂O₂). Bars show mean±s.d.; n>6 wells per group across ≥3 experiments. H₂O₂ (100 μM) reduced VK2 viability relative to control. Post-injury treatment with naïve EVs partially restored viability (vs H₂O₂). BPEV-TRV at 1×10⁶ and 1×10⁸/mL provided greater rescue (###vs. H₂O₂), achieving viability levels at or above the naïve EV group at the same particle dose (*** vs. control).

### 5. Interpretation

Under particle-normalized dosing, BPEV-TRV outperforms naïve AMSC-EVs in (i) promoting VK2 proliferation and (ii) rescuing oxidative damage, consistent with a GSM-relevant epithelial supportive effect. These outcomes align with the physicochemical enhancements (size shift/zeta potential) and miRNA remodeling disclosed for primed AMSC-EVs in Example 2 and Example 5.

Example 5 TRV Activation of hUC-MSCs Produces Extracellular Vesicles with Anti-Catabolic, Anti-Inflammatory miRNA Cargo for Osteoarthritis (OA)

### 1. Purpose and Scope

This example discloses production of BPEV-TRV (TRV-activated hUC-MSC-derived extracellular vesicles) and documents microRNA remodeling pertinent to OA pathology. The disclosure includes cell source and qualification, TRV activation, EV preparation, and miRNA NGS with functional interpretation for cartilage homeostasis, inflammation control, and fibrosis moderation.

### 2. Materials and Reagents

DMEM (Gibco); exosome-depleted FBS (System Biosciences); penicillin-streptomycin; PBS; DMSO; TRV polyphenols [representative: 2,3,5,4′-tetrahydroxystilbene-2-O-β-D-glucoside (THSG) and resveratrol]; 0.22 μm PES filters; TFF system with 100 kDa MWCO module (Cytiva); Illumina small-RNA library kit and flow cells.

### 3. Cell Source and Qualification

Human umbilical cord MSCs (hUC-MSCs) were obtained from an accredited biobank under IRB-compliant consent. Master bank P2; working passages P3-P6 were used. Mycoplasma/sterility tests were negative. Flow cytometry confirmed MSC identity: CD73/CD90/CD105 >95%; CD34/CD45/HLA-DR<2%.

### 4. Culture and TRV Activation

Cells were expanded at 37° C., 5% CO₂ in DMEM+10% exosome-depleted FBS+1% penicillin-streptomycin. At 70-80% confluence, monolayers were rinsed and switched to serum-free DMEM containing TRV polyphenols. A representative condition used THSG 10 μM+resveratrol 5 μM (stocks in DMSO; final solvent≤0.1% v/v) for 24-72 h. Cell viability remained >95% by CCK-8.

### 5. Naïve EV Control

Naïve hUC-MSC EVs were prepared in parallel under identical conditions without TRV. All downstream processing was the same for TRV and naïve preparations.

### 6. EV Harvest and Purification

Conditioned medium (150-200 mL per T175) was processed as follows: 300×g 10 min →2,000×g 20 min→10,000×g 30 min (4° C.); 0.22 μm filtration; TFF (100 kDa MWCO) with PBS diafiltration (5-8 diavolumes), transmembrane pressure 0.5-0.8 bar, cross-flow 25-40 mL/min; concentration factor 10-15×. Retentate was collected as EV suspension for analysis or lyophilized as needed. Particle characterization (e.g., TRPS, zeta potential, CD markers) can be performed as Example 1.

7. miRNA Extraction, Library Preparation, Sequencing, and Statistics

Total RNA from BPEV-TRV and naïve EVs (biological n≥3 per group, where available) was extracted (exoRNeasy or equivalent), assessed on Bioanalyzer 2100, prepared as small-RNA libraries, and sequenced on an Illumina platform (8-12 M reads/sample). Adapter trimming and Phred <30 filtering were applied; reads were mapped to hg38 with hsa-miR annotations. Differential expression was computed by DESeq2 with Benjamini-Hochberg correction; adjusted p<0.05 was considered significant. Fold-change (FC) denotes TRV/naïve on normalized counts.

8. Differential Hsa-miR Profile Relevant to OA (from Table 6; Human Source Only)

TABLE 6
Differential microRNA profile of hUC-MSC-derived BPEV-TRV versus naïve hUC-
MSC EVs, listing read counts, TRV/naïve fold-change, functional significance,
and mechanisms related to chondroprotection, anti-inflammatory activity, ECM homeostasis,
and autophagy. Table 6 shows upregulated regulation supporting cartilage ECM homeostasis,
anti-inflammation, and anti-fibrosis. (TRV/Naïve fold-change >1).

| | Naïve | BPEV-TRV | FC | OA-relevant | Representative |
|---|---|---|---|---|---|
| miRNA | counts | counts | (TRV/Naïve) | significance | mechanism |
| hsa-miR-126-3p | 70 | 1348 | 19.26 | Indirect | Lowering aberrant |
| | | | | (angiogenesis | neovascularization in |
| | | | | moderation) | OA cartilage |
| hsa-miR-146a-5p | 2 | 81 | 40.50 | Direct | IRAK/TRAF |
| | | | | (anti-inflammatory) | suppression; |
| | | | | | anti-inflammatory |
| hsa-miR-140-3p | 22 | 36 | 1.64 | Direct (cartilage | Targets |
| | | | | ECM homeostasis) | ADAMTS/MMP; |
| | | | | | supports |
| | | | | | ACAN/COL2A1 |
| hsa-miR-140-5p | 75 | 116 | 1.55 | Direct (cartilage | Inhibits |
| | | | | ECM homeostasis) | ADAMTS5/MMP13; |
| | | | | | chondrocyte |
| | | | | | maintenance |
| hsa-miR-221-3p | 229 | 320 | 1.40 | Protective | Reduction linked to |
| | | | | down-regulation | lower catabolism |
| | | | | (catabolic) | |
| hsa-miR-199b-5p | 8 | 11 | 1.38 | HIF/mTOR | Anti-inflammatory; |
| | | | | crosstalk | stress adaptation |
| hsa-miR-16-5p | 10,466 | 13,653 | 1.30 | Protective | Relieves BCL2 |
| | | | | down-regulation | repression; enhances |
| | | | | (pro-apoptotic) | survival |
| hsa-miR-29c-3p | 93 | 113 | 1.22 | Anti-fibrotic/ECM | Normalizes matrix |
| | | | | | remodeling |
| hsa-miR-29a-3p | 6331 | 7544 | 1.19 | Anti-fibrotic/ECM | Reduces aberrant |
| | | | | | collagen I deposition |
| hsa-miR-34a-5p | 578 | 675 | 1.17 | Protective | Limits chondrocyte |
| | | | | down-regulation | apoptosis/senescence |
| | | | | (pro-apoptotic) | |
| hsa-miR-199a-5p | 180 | 203 | 1.13 | HIF/mTOR | Stress adaptation; |
| | | | | crosstalk | chondroprotection |
| hsa-miR-210-3p | 77 | 80 | 1.04 | Hypoxia adaptation | Supports phenotype |
| | | | | | under low O₂ |

TABLE 7
Differential microRNA profile of hUC-MSC-derived BPEV-TRV versus naïve hUC-
MSC EVs, listing read counts, TRV/naïve fold-change, functional significance,
and mechanisms related to chondroprotection, anti-inflammatory activity, ECM homeostasis,
and autophagy. Table 7 shows downregulated regulation supporting cartilage ECM homeostasis,
anti-inflammation, and anti-fibrosis. (TRV/Naïve fold-change <1).

| | Naïve | BPEV-TRV | FC | OA-relevant | Representative |
|---|---|---|---|---|---|
| miRNA | counts | counts | (TRV/Naïve) | significance | mechanism |
| hsa-miR-23b-3p | 195 | 186 | 0.95× | TGF-β/SMAD | Damps inflammatory |
| | | | | tuning | signaling; lowers |
| | | | | | MMPs |
| hsa-miR-27b-3p | 117 | 107 | 0.91× | Anti-catabolic | Targets MMP13; |
| | | | | | reduces degradation |
| hsa-miR-155-5p | 609 | 530 | 0.87× | Pro-inflammatory | Reduction lowers OA |
| | | | | | inflammatory damage |
| hsa-miR-199a-3p | 1522 | 1207 | 0.79× | HIF/mTOR | Lowers IL-1β-driven |
| | | | | crosstalk | catabolism |
| hsa-miR-199b-3p | 1522 | 1207 | 0.79× | ECM remodeling | Anti-inflammatory |
| | | | | | trend |
| hsa-miR-24-3p | 573 | 444 | 0.77× | Anti-catabolic | Suppresses MMPs; |
| | | | | | supports survival |
| hsa-miR-98-5p | 2370 | 1661 | 0.70× | Anti-inflammatory | Targets IL-6; mitigates |
| | | | | | signaling |
| hsa-miR-100-5p | 745 | 140 | 0.19× | Autophagy/mTOR | Protective |
| | | | | | recalibration; |
| | | | | | chondroprotection |
| hsa-miR-320a-3p | 20,597 | 3,739 | 0.18× | Anti-inflammatory | Reported to reduce |
| | | | | | MMP13/COX-2 |

Example 6 TRV-Activated AMSC Extracellular Vesicles (BPEV-TRV) Exhibit OA-Relevant microRNA Remodeling

### 1. Purpose and Scope

This example discloses that AMSC-derived EVs produced under TRV bio-pulsing (BPEV-TRV) possess microRNA cargo remodeling associated with anti-catabolic, anti-inflammatory, and matrix-protective effects pertinent to osteoarthritis (OA). Biophysical particle characterization is shared with Example 2; here the emphasis is NGS of gga-miRNAs and OA-focused pathway interpretation.

### 2. Materials and Reagents

As shown in Example 2 (AMSC source): basal AMSC medium, exosome-depleted FBS, penicillin-streptomycin, PBS, DMSO, TRV polyphenols [representative: THSG and resveratrol], 0.22 μm filters, TFF module (100 kDa MWCO), Illumina small-RNA library kit.

### 3. Cell Source and Qualification

AMSCs were established and qualified as in shown Example 2 (mycoplasma/sterility negative; MSC marker pattern confirmed with avian-reactive antibodies). Working passages P3-P6 were used.

### 4. Culture and TRV Activation

Cultures at 70-80% confluence were switched to serum-free medium containing TRV polyphenols (representative: THSG 10 μM+resveratrol 5 μM, final solvent≤0.1% v/v) for 24-72 h. Cell viability remained >95% by CCK-8.

### 5. Naïve EV Control

Matched flasks without TRV served as the naïve AMSC-EV control; downstream processing was identical.

6. EV harvest and purification

Conditioned medium was clarified (300×g, 2,000×g, 10,000×g, 4° C.), filtered (0.22 μm), and purified by TFF (100 kDa MWCO) with 5-8 diavolumes of PBS. Retentates were collected as EV suspensions or lyophilized.

6A. EV Characterization-TRPS (Shared from Example 2)

TRPS (Izon Exoid, NP150) was performed under identical settings for naïve and TRV groups. As disclosed in FIG. 2A of Example 2, naïve AMSC EVs exhibited a mean diameter of ˜110 nm (detected min ˜81 nm, max ˜304 nm), while AMSC-TRV-EVs exhibited a mean diameter of ˜73 nm (min ˜30 nm, max ˜417 nm) with a higher peak and broader higher-diameter tail, confirming preserved small-EV class and enhanced output. No additional particle figures are provided here; FIG. 2A, FIG. 2B, FIG. 3A, FIG. 3B and FIG. 3C from Example 2 apply.

7. miRNA Extraction, Library Preparation, Sequencing, and Statistics (OA Focus)

Total RNA from BPEV-TRV and naïve AMSC-EVs (biological n≥3 per group, where available) was isolated (exoRNeasy or equivalent), quality-checked (Bioanalyzer 2100), prepared as small-RNA libraries, and sequenced on an Illumina platform (8-12 M reads/sample). Reads were adapter-trimmed, quality-filtered (Phred <30 removed), and mapped to the Gallus gallus genome with gga-miR annotations. Differential expression was computed with DESeq2 using Benjamini-Hochberg adjustment (adjusted p<0.05 considered significant). Fold change is defined as TRV/naïve on normalized counts. OA interpretation emphasizes miRNAs implicated in cartilage ECM homeostasis (COL2A1/ACAN), catabolic enzyme regulation (ADAMTS/MMP), inflammation (NF-κB/IL-6), fibrosis (collagen I), hypoxia/angiogenesis, and autophagy/mTOR balance.

### 8. OA-Relevant Differential Gga-miR Profile (Representative)

The following miRNAs were significantly altered in BPEV-TRV versus naïve AMSC-EVs and are associated with OA-pathway modulation:

TABLE 8
Differential microRNA profile of AMSC-derived BPEV-TRV versus naïve AMSC sEVs, listing
read counts, TRV/naïve fold-change, functional significance, and mechanisms linked
to anti-catabolic/ECM-autophagy programs, NF-κB negative feedback, and chondrocyte phenotype
support. Table 8 shows upregulated regulation supporting cartilage protection and survival.

| gga-miRNA | OA relevance | Representative mechanism |
|---|---|---|
| gga-miR-140-5p / -3p | Cartilage ECM homeostasis | Suppresses ADAMTS5/MMP13; supports |
| | | COL2A1/ACAN and chondrocyte phenotype |
| gga-miR-29a-3p/-29c-3p | Anti-fibrotic remodeling | Reduces aberrant collagen I deposition; normalizes |
| | | ECM turnover |
| gga-miR-126-3p | Angiogenesis moderation | Limits aberrant neovascularization in OA cartilage |
| gga-miR-210-3p | Hypoxia adaptation | Supports chondrocyte function under low O₂ |
| gga-miR-199b-5p/-199a-5p | HIF/mTOR crosstalk | Stress adaptation; chondroprotection |

TABLE 9
Differential microRNA profile of AMSC-derived BPEV-TRV versus naïve AMSC sEVs, listing
read counts, TRV/naïve fold-change, functional significance, and mechanisms linked
to anti-catabolic/ECM-autophagy programs, NF-κB negative feedback, and chondrocyte phenotype
support. Table 9 shows protective down-regulation (reduces catabolisminflammation).

| gga-miRNA | OA relevance | Representative mechanism |
|---|---|---|
| gga-miR-155-5p | Pro-inflammatory driver | Reduction lowers OA inflammatory damage |
| gga-miR-98-5p | IL-6 axis | Down-tuning mitigates inflammatory signaling |
| gga-miR-27b-3p /-23b-3p | Catabolic program | Targets MMP13; reduction suppresses cartilage |
| | | degradation |
| gga-miR-24-3p | Anti-catabolic tuning | Lowers MMP expression; supports survival |
| gga-miR-199a-3p /-199b-3p | HIF/mTOR crosstalk | Lowers IL-1β-induced catabolism |
| gga-miR-146a-5p /-146b-5p | NF-κB negative feedback, | Inhibits IRAK1/TRAF6 signaling; reduces |
| | suppressor-type | IL-1β/TNF-α outputs |
| gga-miR-320a-3p | Anti-inflammatory | Reported to reduce MMP13/COX-2 in chondrocytes |
| gga-miR-100-5p | Autophagy/mTOR re-set | Protective recalibration; chondroprotection |

### 9. Pathway Enrichment Overview (KEGG/GO; OA Focus)

Enrichment analysis of significantly altered gga-miRs revealed over-representation of:
(i) Cartilage ECM homeostasis (ACAN/COL2A1 support; ADAMTS/MMP suppression);
(ii) Inflammation control (NF-κB/IL-6 axis moderation; IRAK/TRAF down-tuning);
(iii) Anti-fibrotic remodeling (miR-29 family);
(iv) Autophagy-mTOR balance (miR-99/100, miR-140 clusters); and
(v) Hypoxia/angiogenesis adaptation (miR-210/126 families).

The combined directionality indicates a net anti-catabolic and matrix-preserving EV cargo in BPEV-TRV relative to naïve AMSC-EVs.

Example 7 Head-to-head chondrocyte evaluation of BPEV-TRV from hUC-MSCs and from AMSCs under particle-matched dosing (C2812 human chondrocytes)

### 1. Purpose and Scope

This example compares BPEV-TRV (TRV-activated extracellular vesicles) produced from human umbilical cord MSCs (hUC-MSCs) and from avian MSCs (AMSCs) in the same human chondrocyte model (C2812 cells). Source-matched naïve EVs serve as controls. Doses are normalized by particle number to permit direct potency comparisons. Outcomes include proliferation/viability, migration (scratch closure), and inflammatory/catabolic gene responses to lipopolysaccharide (LPS).

### 2. Materials

Cells: C2812 human chondrocytes (authenticated, mycoplasma-free).

Media: Chondrocyte growth medium (DMEM/F-12+10% exosome-depleted FBS+1% penicillin/streptomycin). For cytokine challenges, use 1% FBS.

TRV activation: Botanical polyphenols (representative condition: THSG 10 μM+resveratrol 5 μM, final solvent≤0.1% v/v) as described previously for each source.

EV preparation: Conditioned media clarified (300×g, 2,000×g, 10,000×g), filtered (0.22 μm), and polished by TFF (100 kDa MWCO); lyophilized and reconstituted in PBS.

Reagents: CCK-8; LPS (E. coli O111: B4; Sigma); Griess reagent; qRT-PCR kits/primers; antibodies for COL2A1, MMP-1, iNOS, COX-2; Annexin V/PI (optional).

Wound-healing consumable: ibidi Culture-Insert 2 Well (500 μm gap), sterile silicone insert for reproducible cell-free gaps in adherent monolayers (ibidi GmbH).

### 3. Study Design and Dosing

C2812 cells were seeded at 2-5×10⁴ cells/cm² and allowed to reach ˜80% confluence at 37° C., 5% CO₂. For proliferation assays, cells received vehicle, naïve EVs (1×10⁷ particles/mL), or BPEV-TRV at 1×10⁷ or 1×10⁹ particles/mL for 48 h (source indicated per group).

For inflammatory injury, LPS (E. coli O111: B4; Sigma) dose-response (0.1, 1, 10 μg/mL, 24 h, 1% FBS) established 1 μg/mL as the challenge level used for rescue experiments. Cells were exposed to LPS 1 μg/mL for 24 h in 1% FBS, then treated for 24 h with vehicle, source-matched naïve EVs (1×10⁷/mL), or BPEV-TRV (1×10⁷ or 1×10⁹/mL).

Wound healing (ibidi Culture-Insert 2 Well, 500 μm).

The ibidi Culture-Insert 2 Well was placed on a clean, flat surface of 24-well plates according to the manufacturer's instructions, creating two reservoirs separated by a 500 μm wall. C2812 cells were seeded into both reservoirs at 2-4×10⁴ cells per reservoir and cultured at 37° C., 5% CO₂ until confluent. The insert was removed vertically with sterile forceps to generate a precisely defined, cell-free gap (no residual cells beneath the walls). Wells were rinsed once with PBS to remove debris and immediately overlaid with treatment medium:

Vehicle; source-matched naïve EVs (1×10⁷ particles/mL); or BPEV-TRV (1×10⁷ or 1×10⁹ particles/mL).

For inflammatory conditions, monolayers were first exposed to LPS 1 μg/mL for 24 h; the insert was then removed and EV treatments applied as above.

### 4. Readouts and Analysis

Proliferation/viability: CCK-8, reported as % of untreated control. A separate LPS dose-response (0.1, 1, 10 μg/mL) established the challenge level (1 μg/mL).

Migration: scratch width quantified at 24 h; percent closure relative to time 0. qRT-PCR (18S reference): TNF-α, IL-1β, IL-6, iNOS, COX-2, COL2A1; MMP-1.

TABLE 10
The sequence of primers for QPCR (Human)

| Gene | | |
|---|---|---|
| name | Forward | Reverse |
| NFKBIA | GGACGAGCTGCCCTATGATG | TTTCAGCCCCTTTGCACTCA |
| TLR2 | GTGTTGCAAGCAGGATCCAA | CCAGTGCTTCAACCCACAAC |
| | A | |
| iNOS | CGTGGAGACGGGAAAGAAGT | GCTGCCCCAGTTTTTGATCC |
| COX2 | TGCGGGAACACAACAGAGTA | AACAACTGCTCATCACCCCA |
| COL2A1 | AGACGTGAAAGACTGCCTCA | TTGGTCCTGGTTGCCCACT |
| | G | |
| MMP1 | TCACACCTCTGACATTCACC | TTGTCCCGATGATCTCCCCT |
| | A | |
| MMP13 | TTGGTCCGATGTAACTCCTC | AGAAGTCGCCATGCTCCTTA |
| | TG | AT |
| IL-1β | GCAGCCATGGCAGAAGTACC | AGTCATCCTCATTGCCACTG |
| | | TAAT |
| TNF-α | TAGCCCATGTTGTAGCAAAC | TTATCTCTCAGCTCCACGCC |
| | CC | A |
| IL-6 | ACCCCCAGGAGAAGATTCCA | GATGCCGTCGAGGATGTACC |
| 18s | GTAACCCGTTGAACCCCATT | CCATCCAATCGGTAGTAGCG |

Statistics: n>6 wells per condition; >3 independent experiments. One-way ANOVA with prespecified multiple comparisons; significance thresholds denoted on figures.

### 5. Results (Dose-Matched by Particle Count)

### 5.1 Proliferation

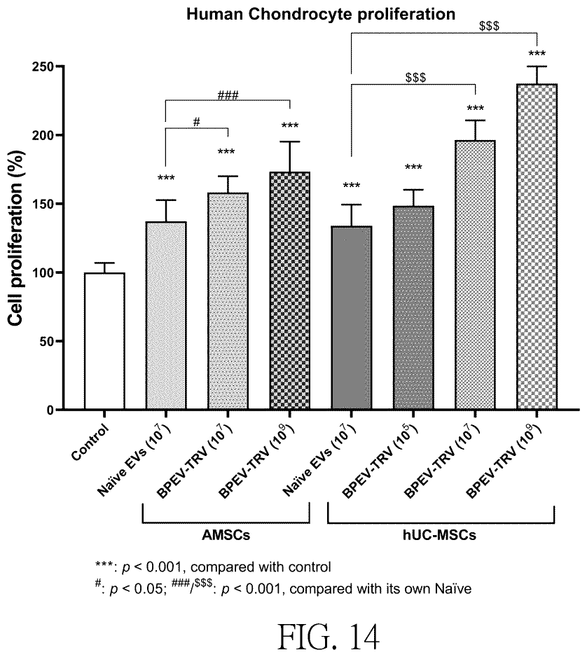

As shown in FIG. 14, CCK-8 after 48 h exposure to vehicle, naïve EVs (1×10⁷/mL), or BPEV-TRV (1×10⁷, 1×10⁹/mL) from AMSCs or hUC-MSCs. Bars show mean±s.d.; *** p<0.001 vs. control; #/ ###and $$$ denote comparisons vs. the corresponding naïve EV within source, as indicated. Naïve EVs (1×10⁷/mL) increased C2812 proliferation above vehicle. BPEV-TRV showed dose-dependent enhancement, with both AMSC-BPEV-TRV and hUC-MSC-BPEV-TRV exceeding their respective naïve EVs at 1×10⁷ and further at 1×10⁹ particles/mL. At equal particle numbers, hUC-MSC-BPEV-TRV displayed the highest proliferation gain among all groups.

### 5.2 LPS Cytotoxicity and Rescue

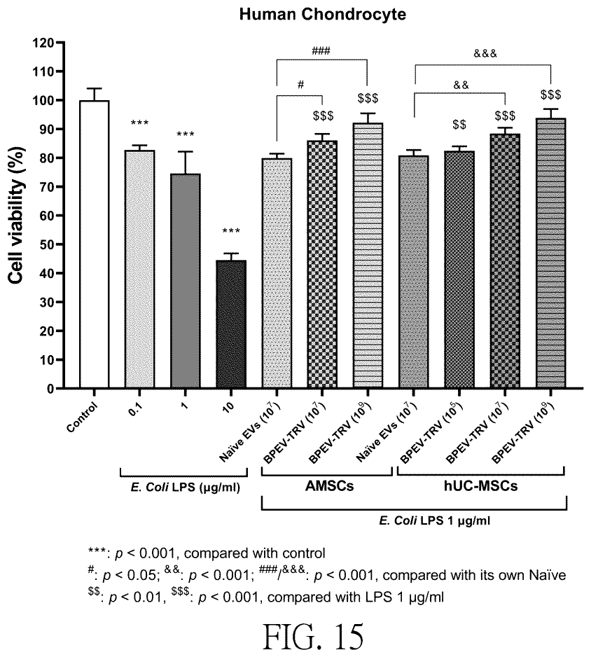

As shown in FIG. 15, on left: LPS dose-response (0.1, 1, 10 μg/mL), on right: rescue at LPS 1 μg/mL after 24 h, followed by 24 h treatment with source-matched naïve EVs (1×10⁷/mL) or BPEV-TRV (1×10⁷, 1×10⁹/mL). Symbols indicate significance vs. control and vs. LPS, as annotated. LPS suppressed viability in a dose-dependent manner (0.1, 1, 10 μg/mL), most prominently at 1 μg/mL and 10 μg/mL. At 1 μg/mL, post-challenge treatment with naïve EVs partially restored viability, whereas BPEV-TRV from both sources produced greater rescue, with 1×10⁹/mL approaching or surpassing unchallenged levels; 1×10⁷/mL also improved viability relative to LPS alone.

5.3 Migration (Ibidi Culture-Insert Wound-Healing Assay) with and without LPS

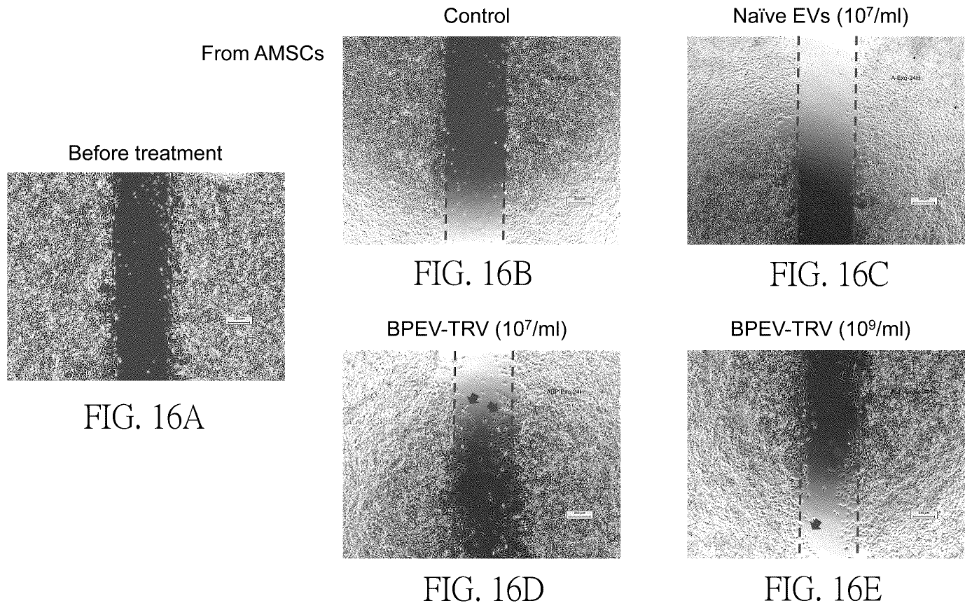

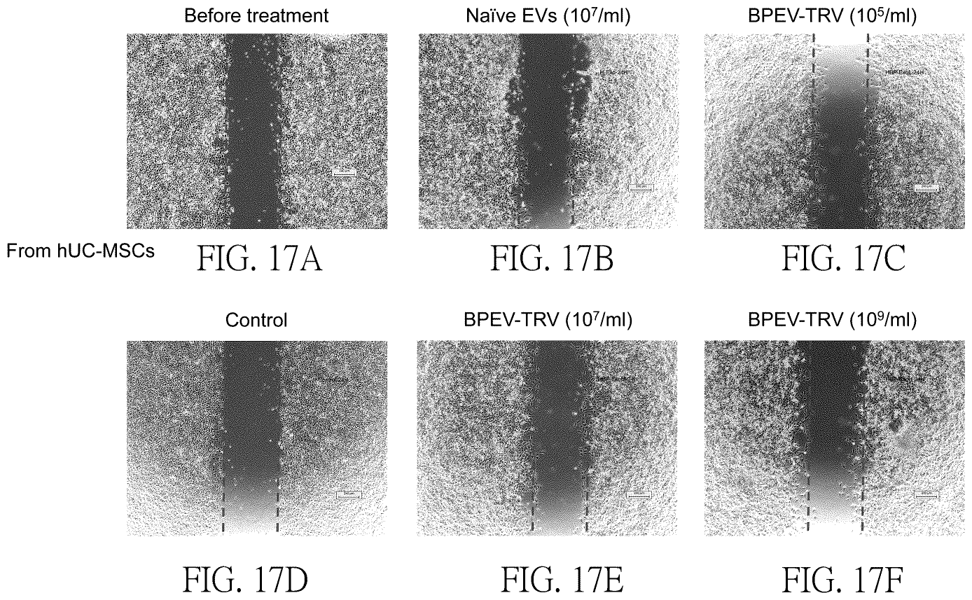

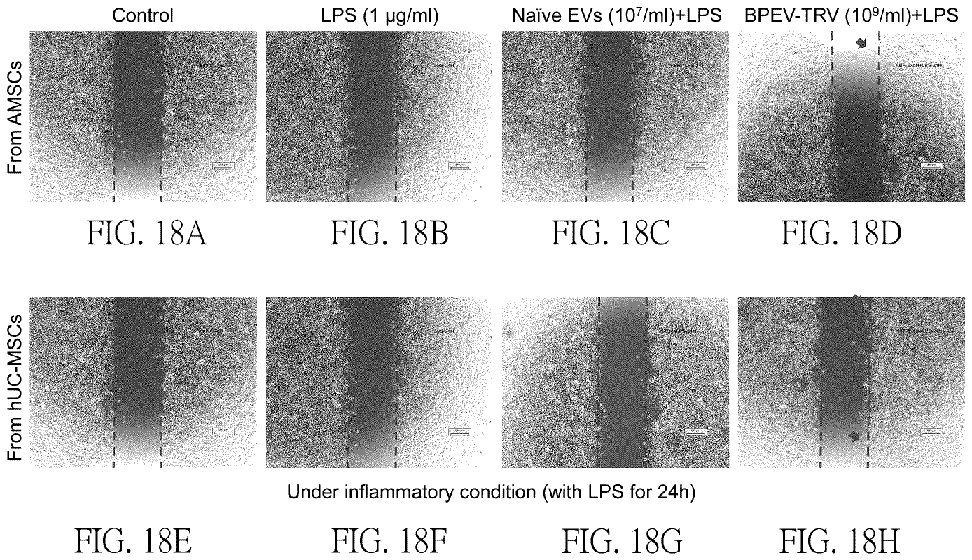

Using the standardized 500 μm ibidi gap, both AMSC-BPEV-TRV and hUC-MSC-BPEV-TRV accelerated closure at 1×10⁷ and 1×10⁹ particles/mL versus vehicle and their respective naïve EVs. Under LPS 1 μg/mL, BPEV-TRV retained pro-migratory activity and achieved significantly greater % Closure than naïve EVs at matched particle numbers. As shown in FIG. 16A to FIG. 16E, C2812 monolayers established with the Culture-Insert 2 Well (500 μm); images at 0 h and 24 h. Red dashed lines indicate the original gap edges; % Closure quantified as described. AMSC-derived BPEV-TRV enhances chondrocyte migration in the ibidi Culture-Insert assay. As shown in FIG. 17A to FIG. 17F, hUC-MSC-derived BPEV-TRV enhances chondrocyte migration in the ibidi Culture-Insert assay. As shown in FIG. 18A to FIG. 18H, following LPS 1 μg/mL, 24 h, the insert was removed and EVs applied; BPEV-TRV (1×10⁹/mL) outperformed naïve EVs (1×10⁷/mL) for both sources at 24 h, the images show migration under inflammatory conditions (ibidi Culture-Insert).

### 5.4 Inflammatory Cytokines

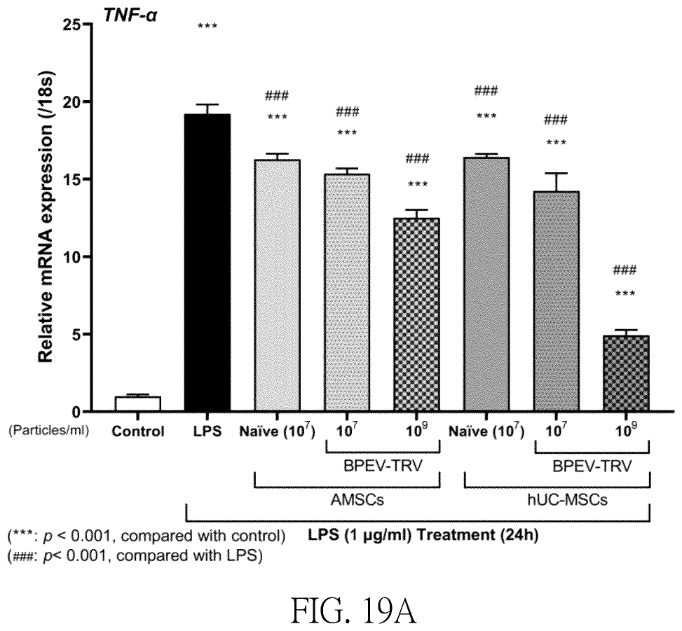

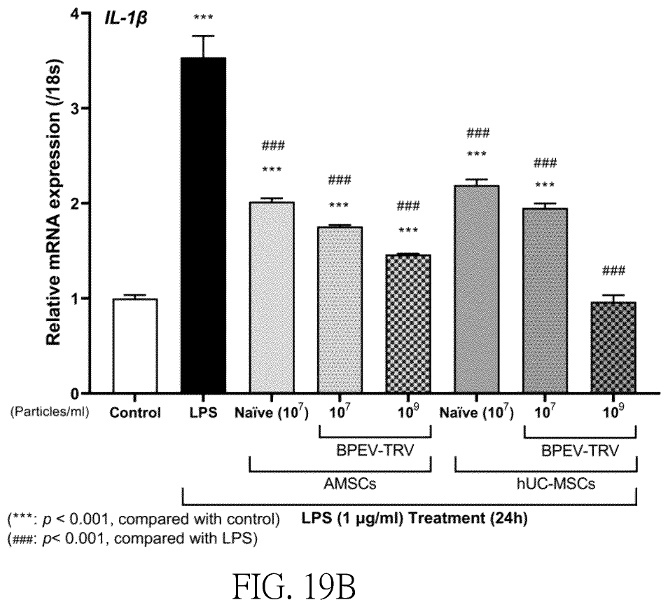

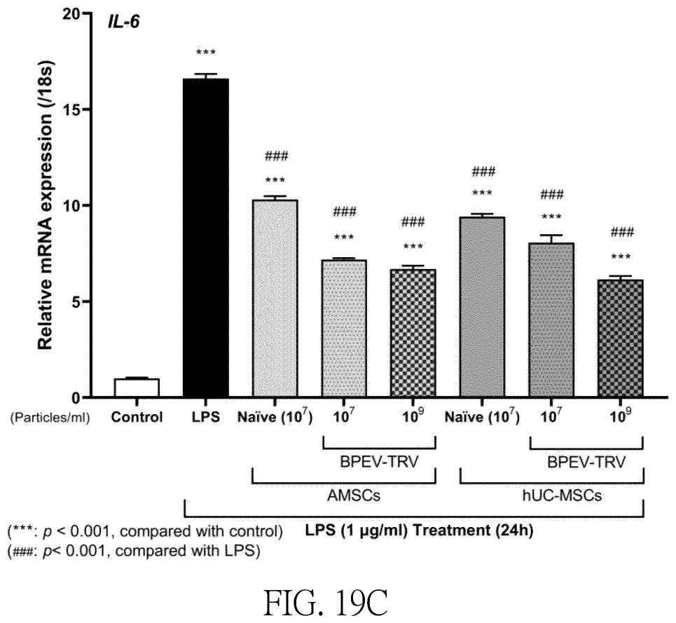

As shown in FIG. 19A to FIG. 19C, qRT-PCR of TNF-α, IL-1β, IL-6 after LPS 1 μg/mL, 24 h, followed by 24 h EV treatment. Data normalized to 18S; *** p<0.001 vs. control; ###p<0.001 vs. LPS. LPS elevated TNF-α, IL-1B, IL-6 transcripts. BPEV-TRV reduced all three markers in a dose-responsive manner for both sources; 1×10⁹/mL yielded the largest suppression and was consistently superior to matched-dose naïve EVs.

5.5 iNOS and COX-2

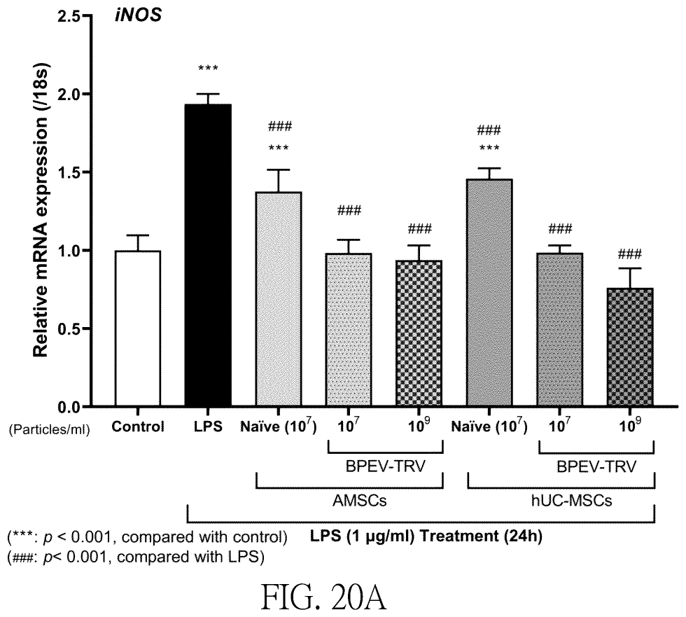

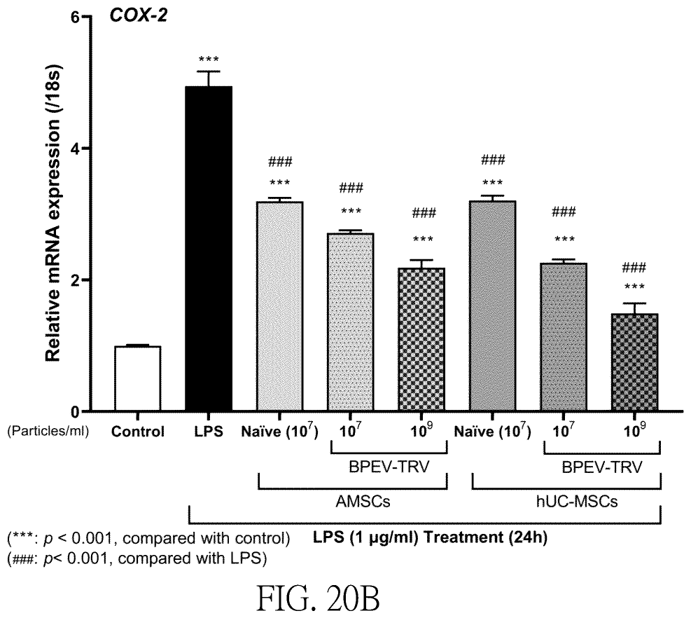

As shown in FIGS. 20A and 20B, LPS strongly induced iNOS and COX-2. BPEV-TRV suppressed both targets at 1×10⁷ and 1×10⁹/mL; suppression at 1×10⁹/mL was greater than naïve EVs at 1×10⁷/mL for each source.

### 5.6 Matrix Balance-COL2A1 and MMP-1

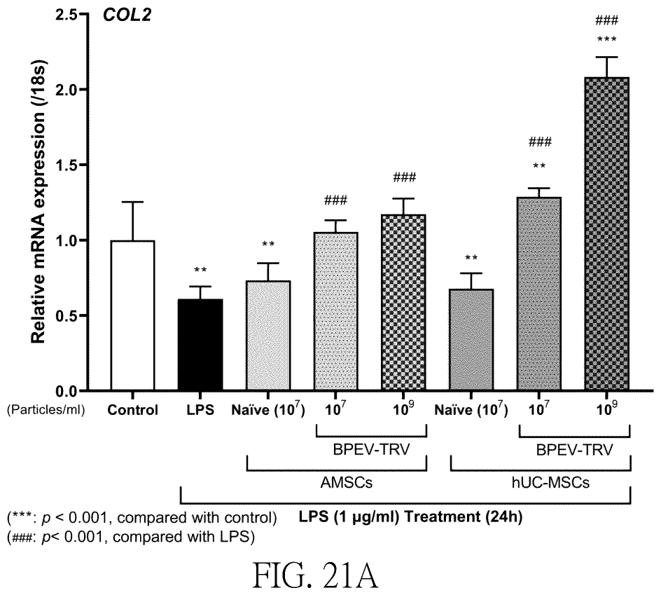

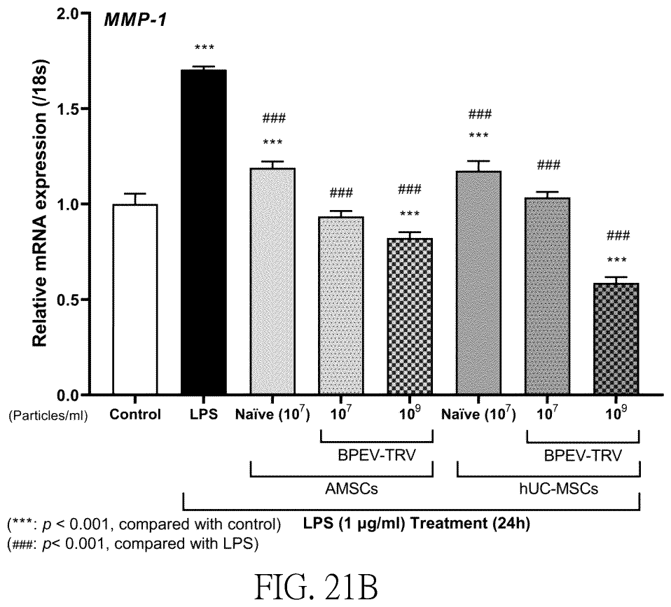

As shown in FIGS. 21A and 21B, LPS depressed COL2A1 and increased MMP-1. BPEV-TRV reversed these trends: COL2A1 rose above LPS+vehicle and above naïve EVs, and MMP-1 decreased to near-baseline or below naïve levels in a dose-dependent fashion. hUC-MSC-BPEV-TRV (1×10⁹/mL) showed the largest COL2A1 increase among all groups.

### 6. Interpretation

Across proliferation, viability, migration, inflammatory mediators, and matrix markers, BPEV-TRV from both AMSCs and hUC-MSCs outperformed their respective naïve EVs at particle-matched doses. The combined effects-reduced TNF-α/IL-1β/IL-6, iNOS, COX-2, lowered MMP-1, and increased COL2A1—are consistent with anti-inflammatory, anti-catabolic, and pro-repair activities valuable for osteoarthritic cartilage.

The present disclosure has been described with embodiments thereof, and it is understood that various modifications, without departing from the scope of the present disclosure, are in accordance with the embodiments of the present disclosure. Hence, the embodiments described are intended to cover the modifications within the scope of the present disclosure, rather than to limit the present disclosure. The scope of the claims therefore should be accorded the broadest interpretation so as to encompass all such modifications.

What is claimed is:

1. A composition comprising primed extracellular vesicles and a biologically or pharmaceutically acceptable carrier, wherein the primed extracellular vesicles comprise an upregulated miRNA, and a fold change of the upregulated miRNA of the primed extracellular vesicles compared to miRNA of naturally occurring extracellular vesicles is greater than 1.

2. The composition of claim 1, wherein the primed extracellular vesicles are cultivated from mesenchymal stem cells.

3. The composition of claim 2, wherein the mesenchymal stem cells are selected from the group consisting of avian mesenchymal stem cells, human umbilical cord mesenchymal stem cells, human placental mesenchymal stem cells, adipose-derived mesenchymal stem cells, bone-marrow mesenchymal stem cells, and a combination thereof.

4. The composition of claim 1, wherein the fold change of the upregulated miRNA of the primed extracellular vesicles compared to the miRNA of the naturally occurring extracellular vesicles is greater than 1, and the primed extracellular vesicles are cultivated from avian mesenchymal stem cells or from human mesenchymal stem cells, and the upregulated miRNA of the primed extracellular vesicles is selected from the group consisting of miR-22-3p, miR-34a-5p, miR-132a-3p, miR-132a-5p, miR-140-3p, miR-140-5p, miR-143-3p, miR-145-5p, miR-146a-5p, miR-146b-5p, miR-146c-5p, miR-181a-5p, miR-181b-5p, miR-199-3p, miR-199-5p, miR-210-3p, miR-210a-5p, miR-212-5p, miR-221-3p, and a combination thereof.

5. The composition of claim 4, wherein the upregulated miRNA of the primed extracellular vesicles is selected from the group consisting of miR-22-3p, miR-143-3p, miR-181a-5p, miR-181b-5p, and a combination thereof.

6. The composition of claim 5, wherein the fold change of the upregulated miRNA of the primed extracellular vesicles compared to the miRNA of the naturally occurring extracellular vesicles is greater than 2, and the upregulated miRNA of the primed extracellular vesicles is selected from the group consisting of miR-143-3p, miR-181a-5p, miR-181b-5p, and a combination thereof.

7. The composition of claim 6, wherein the fold change of the upregulated miRNA of the primed extracellular vesicles compared to the miRNA of the naturally occurring extracellular vesicles is greater than 3, and the upregulated miRNA of the primed extracellular vesicles is selected from the group consisting of miR-181a-5p, miR-181b-5p, and a combination thereof.

8. The composition of claim 1, wherein a fold change of a downregulated miRNA of the primed extracellular vesicles compared to miRNA of naturally occurring extracellular vesicles is less than 1.

9. The composition of claim 8, wherein the downregulated miRNA of the primed extracellular vesicles is selected from the group consisting of miR-15a-5p, miR-16-5p, miR-16-1-3p, miR-21-5p, miR-26a-5p, miR-29a-3p, miR-29c-3p, miR-34a-5p, miR-126-3p, miR-142-3p, miR-142-5p, miR-155-5p, miR-182-5p, miR-199a-5p, miR-199b-5p, miR-200a-3p, miR-200b-3p, and a combination thereof.

10. The composition of claim 1, wherein the extracellular vesicle is an apoptotic body, a microvesicle, an exosome, or any combination thereof.

11. The composition of claim 1, wherein the extracellular vesicles comprise a polydisperse population of particles with a diameter ranging from about 30 nm to about 200 nm.

12. The composition of claim 1, wherein the composition is formulated into an injection solution, an intra-articular suspension, a vaginal gel, a hydrogel, or a scaffold composition.

13. A method for preventing, treating, or ameliorating a gynecological or a rheumatological disorder comprising administering the composition of claim 1 to a subject in need thereof.

14. The method of claim 13, wherein the gynecological disorder is selected from the group consisting of genitourinary syndrome of menopause (GSM), premature ovarian insufficiency (POI), ovarian dysfunction, uterine atrophy, and vaginal epithelial thinning.

15. The method of claim 13, wherein the rheumatological disorder is selected from the group consisting of osteoarthritis (OA), rheumatoid arthritis (RA), systemic lupus erythematosus (SLE), psoriatic arthritis, and Sjögren's syndrome.

16. The method of claim 13, wherein the administering comprises topical administration.

17. The method of claim 13, wherein the administering comprises local administration.

18. The method of claim 13, wherein the composition reduces chronic inflammation, oxidative stress, immune dysregulation, and/or progressive tissue degeneration.

19. A method for producing the composition of claim 1, comprising:
cultivating a stem cell in a culture medium containing a Polygonum multiflorum Thunb extract and resveratrol to obtain a cell culture;
obtaining the extracellular vesicle from the cell culture; and
mixing a biologically or pharmaceutically acceptable carrier with the extracellular vesicle.

20. The method of claim 19, wherein the stem cell is mesenchymal stem cell.

21. The method of claim 20, wherein the mesenchymal stem cell is selected from the group consisting of avian mesenchymal stem cell, human umbilical cord mesenchymal stem cell, human placental mesenchymal stem cell, adipose-derived mesenchymal stem cell, bone-marrow mesenchymal stem cell, and a combination thereof.

22. The method of claim 19, wherein the Polygonum multiflorum Thunb extract comprises 2,3,4′,5-tetrahydroxystilbene-2-O-β-D-glucoside (THSG).

23. The method of claim 19, wherein the Polygonum multiflorum Thunb extract has a concentration of about 0.1 μM to about 50 μM.

24. The method of claim 19, wherein the cultivation of the stem cell is performed for a time period ranging from 24 hours to 96 hours.

25. The method of claim 19, wherein the composition is formulated into an injection solution, an intra-articular suspension, a vaginal gel, a hydrogel, or a scaffold composition.
